(19) United States
(12) Patent Application Publication
(10) Pub. No.: US 2025/0236913 A1
(43) Pub. Date: Jul. 24, 2025

(54) NEW METHOD TO DIAGNOSE INFLAMMATORY DISEASES

(71) Applicants: INSTITUT NATIONAL DE LA SANTÉ ET DE LA RECHERCHE MÉDICALE, Paris (FR); UNIVERSITÉ DE MONTPELLIER, Montpellier (FR); INSTITUT RÉGIONAL DU CANCER DE MONTPELLIER, Montpellier (FR)

(72) Inventors: Alain THIERRY, Montpellier (FR); Brice PASTOR, Montpellier (FR); Ekaterina PISAREVA, Montpellier (FR)

(21) Appl. No.: 18/293,571

(22) PCT Filed: Aug. 5, 2022
(86) PCT No.: PCT/EP2022/072147

§ 371 Date: Jan. 30, 2024

(30) Foreign Application Priority Data
Aug. 6, 2021 (EP) 21306103.9

(51) Int. Cl. C12Q 1/6883; C12Q 1/6886; G01N 33/564; G01N 33/569; G01N 33/574

(52) U.S. Cl. CPC C12Q 1/6883; C12Q 1/6886; G01N 33/564; G01N 33/56983; G01N 33/57419; C12Q 2600/156; G01N 2333/165; G01N 2333/908; G01N 2333/966

(57) ABSTRACT

The present invention relates to the diagnostic of inflammatory diseases. The inventors described methods using NET biomarkers as diagnostic biomarkers for inflammatory diseases. COVID-19, Lupus or mCRC are used here as illustrative models for investigating an inflammatory disease. Examples in highlighting variation of the respective correlation of NET biomarkers in this invention rely on the determination of the NET main constituents: (i), DNA as determined by examining the amount of circulating DNA (cirDNA) that corresponds to the amount of NET as being degradation by-products that are released into the circulation; (ii) NE; and (iii), MPO; as well as the detection of a blood compound being indirectly associated to NET formation like the anti-cardiolipin auto-antibody. The invention provides threshold values of NE, MPO, cir-nDNA, and cir-mtDNA blood concentrations and of MNR that can be combined to diagnose/screen individuals. Thus the invention relates to a method for diagnosing a subject for an inflammatory disease comprising the steps of i) determining in a sample obtained from the subject the level of at least one marker selected in the group consisting in NET protein markers, cir-nDNA, cir-mtDNA and/or a cir-DNA fragmentation index.

### FIELD OF THE INVENTION

The present invention relates to a method for diagnosing a subject for an inflammatory disease comprising the steps of i) determining in a sample obtained from the subject the level of at least one marker selected in the group consisting in NET protein markers, cir-nDNA, cir-mtDNA and/or a cir-DNA fragmentation index.

### BACKGROUND OF THE INVENTION

Inflammation is the reaction of the immune system to external (i.e. infection) or internal aggression (i.e. illness). Inflammation concerns all tissues, involving innate immunity and adaptive immunity. But sometimes inflammation can be misdirected and the immune system can attack healthy tissue instead. Thus, inflammatory diseases is when the immune system attacks the body's own tissues, resulting in inflammation. In some cases, our immune system triggers inflammation when there are no invaders to fight off. In these autoimmune diseases, our immune system acts as if regular tissues are infected or somehow unusual, causing damage. Conditions linked to chronic inflammation include: Inflammatory bowel disease, Multiple sclerosis, Psoriasis, Asthma, Rheumatoid arthritis, Obesity, Fatty liver diseases, endometriosis, Type 1 and 2 diabetes mellitus, Alzheimer's and Parkison's disaeses, Vasculitis, Cancer; autoimmune diseases such as Systemic Lupus erythematous.

The inventors postulate that these inflammatory disorders are at least partly, due to a dysregulation of NETs formation resulting in exacerbation of NETs (NETopathies).

Cancer can be also considered as an inflammatory disease. Numerous studies have elucidated the role of circulating neutrophils, circulating NETs and circulating NETs by-product in cancer. It has been demonstrated that various cancer types such as breast, lung or colorectal cancer exhibit an increase in circulating neutrophil numbers (29). There is currently an exponential growth in the literature reporting the emerging role of NETs in tumor progression and metastasis (30; 31; 32; 33). CRC is one of the malignant diseases which shows the greatest involvement of NETs in tumor progression and metastasis (32;33). It appears that, by sequestering circulating tumor cells in NETs, neutrophils fertilize the pre-metastasis niche. Furthermore, cancers predispose neutrophils to release extracellular DNA traps, which contribute to cancer-associated thrombosis (34).

December 2019 saw the emergence of the severe acute respiratory syndrome-Coronavirus 2 (SARS-CoV-2), which causes the coronavirus disease-2019 (COVID-19). Several clinical syndromes associated with SARS-CoV2 are described: asymptomatic forms, uncomplicated disease, non-severe pneumonia and severe pneumonia, acute respiratory distress syndrome (ARDS), a life-threatening respiratory failure, and also sepsis and septic shock with multivisceral failure syndrome. Patients with COVID-19 display polymorphic manifestations including clinical features like fever, nonproductive cough, dyspnea, myalgia, fatigue, with paraclinical characteristics like normal or decreased leukocyte counts, and radiographic evidence of pneumonia. Accumulating evidence reveals that an excessive and uncontrolled release of pro-inflammatory cytokines, called cytokine storm, occurs frequently in severe cases. This cytokine storm leads to ARDS, multiple organ damage, and even death. The COVID-19 cytokine storm is also clearly characterized in critically ill patients by substantial impairment of the host immune system and, in particular, the innate immune response (8,9).

Neutrophils play an important role as the first line of innate immune defense. One of their functions known as neutrophil extracellular traps (NETs) was discovered in 2004 (10). These are extensive structures released extracellularly from activated neutrophils in response to infection. They are composed of granular protein assembled on a scaffold of released chromatin. These structures impede the dissemination of microorganisms in blood by trapping them mechanically, and by exploiting coagulant function to segregate them within the circulation. NET components (DNA, histones, granule proteins) also contribute to the triggering of an inflammatory process (1,11,12,13). While extracellular traps (ETs) formation is prominent in neutrophils, several other types of innate or adaptative immune cells reportedly release, following strong activation signals, chromatin and granular proteins (MPO, NE, . . . ) into the extracellular space, forming ETs: macrophages, cosinophils, basophils, mast cells and lymphocytes (11,14).

NET function, however, can be considered a “double-edged sword” (12). On one hand, as an innate immune response, NET formation is an efficient strategy for neutralizing invasive microorganisms. On the other hand, NET can be harmful to the host, in that its exposed by-products are toxic to endothelial cells and parenchymal tissue. Unbalanced NET formation and neutrophil activation may therefore play a significant role in the pathogenesis of numerous non-autoimmune pathologies, such as thrombosis, cystic fibrosis, sepsis, transfusion-related acute lung injury, severe obesity, gouty arthritis, pre-eclampsia or kidney diseases; and in the pathogenesis of autoimmune diseases such as lupus, type 1 diabetes, vasculitis or rare conditions affecting small blood vessels, particularly those of the lungs, skin and kidneys (11,12).

There are various approaches to controlling NET formation in the context of viral infection. Naturally-occurring desoxyribonuclease I (DNase-1) digests extracellular chromatin and NETs (12,11). Low level bioactivity of endogenous DNase-1 may lead to a dysregulation of NETs, thus causing autoimmune diseases and other inflammatory disorders. DNase-1 is the only NET-targeting molecule already in use in clinical practice, as it is used to treat both cystic fibrosis in order to improve lung function and reduce infectious exacerbations, and virus-associated bronchiolitis. However, the fact that DNase-1 dismantles the NET structure without degrading the whole protein components of NETs, indicates that it is less effective in abrogating a NET-triggered inflammatory response. The latter can be targeted with using histone-blocking antibodies (15). As regards neutrophil/platelet interactions, aspirin treatment decreases NET formation in the lung microcirculation and plasma, and also decreases the deposition of platelets with neutrophils on lung vascular walls (15). Very different structural classes of molecules can inhibit the potent neutrophil stimulus for the release of NETs by platelet activation of endosomal toll-like receptors (TLRs). Such approaches include anti-CLEC (C-type Lectin-like receptors) and especially a bispecific anti-CLEC5A/TLR2 monoclonal antibody (16). Hydroxychloroquine, a broadly anti-malarial and anti-inflammatory drug, shows TLR-pathway blockage capacity (17). The use of biologics to block cytokines is now widespread, as in the use of newer, small molecule drugs such as ‘Jakinibs’ (18), or anti-interleukin 6 (IL-6) approaches to block neutrophil function18. Self-DNA re-entry may be recognized by TLR

DNA sensors as damage-associated molecular patterns (DAMPs) (11).

### Link between COVID-19 and NETs Dysregulation

The inventors were among the first (1,4-7) to flagg the analogous biological and physiological features of COVID-19 infection and the detrimental amplification loop between inflammation and tissue damage induced by NETosis dysregulation (2-4). Widely described, both are complex diseases which result in inflammatory processes and multi-organ damages. More specifically, both are associated with an abnormality of coagulation factors, prothrombotic activity, and with cytoxicity towards endothelial and epithelial cells, leading in particular to systemic vascular permeability. As a result of this, vasculitis, myocardial infarction, hemorrhage or systemic side effects on the blood supply and on the functions of multiple organs are observed in both disorders (4). With respect to biologics, their effects include overconcentration of neutrophils in lung vascularization and high levels of interferon, C reactive protein, lactate deshydrogenases, proinflammatory cytokines, and high amount of circulating fibrinogen. Accordingly, both may lead to failure of respiration function to the extent of ARDS, and also thrombosis, sepsis, acute cardiac injury and heart failure (1,4).

More, high levels of circulating NETs (and related increased amounts of circulating DNA and histones) are detected in patients with viral infections such as hantavirus or human immunodeficiency virus (HIV). NETs and neutrophils are also involved in the pathologies of chikungunya virus, simian immunodeficiency virus, influenza, parvovirus, rhinovirus and influenza-associated pneumonia (13,15).

### A Link between COVID-19 Physiopathology and NETopathy

Based on the correlations of COVID-19 symptoms with those consecutive to uncontrolled NET formation in various sterile or infectious diseases, the inventors were the first to postulate that COVID-19 induces a disproportionate virus-induced NET release, which plays a key role in COVID-19 pathogenesis (1,4).

Viruses are known for their extraordinary capacity to evade immune control mechanisms. The inventors hypothesized that viral mechanisms target NET formation by impairing the clearance of NET and extracellular DNA. This would lead to harmful positive amplification of virally-driven hyperinflammation. This finding suggests a significant new direction for the development of treatments for this acute viral infection. Investigation of NETosis and the pathogenesis of extracellular DNA is all recent, with the result that research and development on treatments that would inhibit the amplifier loop induced by unbalanced NETosis also remains in its infancy. While neutrophils are the principal starting point for DNA release, targeting NETs rather than neutrophils themselves may in practice be a preferable strategy.

Two main phases might be considered in the progression of COVID-19: at the appearance of the first symptoms, and again at the start of the cytokine storm featuring respiratory failure. While antivirals can be administered throughout the course of the illness, the use of immunomodulators appears less beneficial and even counterproductive, at the point where the immune response is exacerbated, possibly due to NETosis' “double-edged sword” effect. COVID-19 mild disease should feature an early onset of inflammatory reactions with elevated local or systemic vascular permeability, before the adaptive immune system is fully activated, highlighting the significant contribution of the innate immune response.

The inventors highlighted the possible link between the Kawasaki Disease-like syndrome found in a small number of COVID-19 positive children and the exacerbation of a function of the innate immune response, namely the formation of NETs, which can cause systemic vasculopathy and heart failure (19). Yoshida et al (22) recently reported that spontaneous NET formation was enhanced in neutrophils from patients with acute KD. This observation associates KD with NETs formation and supports our hypothesis that NETs and NET by-products play a key role in COVID-19 pathogenesis (19). The inventors report highlighted the likely contribution of the dysregulation of NET formation in COVID-19 pathogenesis and may indicate that the occurrence of this syndrome in children is a signal of COVID-19. The possible link between KD like syndrome and COVID-19, illustrates the obvious contribution of coagulopathy in COVID-19 pathogenesis.

The rare “KD like” syndrome in these children might highlight host/genetic factors effecting COVID-19 individual susceptibility. The ability to decipher individual predispositions to SARS-CoV-2 infection or severe illness, in light of variations in host immunological and inflammatory responses, in particular due to genetic variations, would be of great benefit in infection management. To this end, the inventors recently associated, for the first time, the description of COVID-19 clinical complications, comorbidities, environmental and genetic factors (7). The inventors also give examples of underlying genomic susceptibility to COVID-19, especially with regard to the newly reported link between the disease and the unbalanced formation of neutrophil extracellular traps. As a consequence, the inventors propose that the host/genetic factors associated with COVID-19 call for precision medicine in its treatment (7).

NETs have been linked with severe infections, such as sepsis, and may serve as an additional defense of the innate immune system against circulating microorganisms, including bacteria, Fungi, protozoa, and viruses. Conservation of the NET function across species suggests an evolutionary advantage of NETs in immune defense. The formation of NETs was originally described as a new cell death program, different from apoptosis and necrosis. This “classic” NET formation is dependent on the oxidative explosion and leads to the release of NETs by 20 to 60% of human neutrophils, after 2 to 4 h of stimulation with microorganisms or activators of protein kinase C (PKC), such as phorbol myristate acetate (PMA). During this process, the histones are cleaved by the elastase derived from the granulations, and citrullinated by peptidyl arginine deiminase 4 (PAD4). This process leads to decondensation and sagging of the chromatin. The combined rupture of the nuclear and granular membranes leads to the mixing of the cytoplasmic, granular and nuclear components. The rupture of the plasma membrane then allows the release of NETs in the extracellular space. Initially, neutrophil elastase degrades the linker histone protein H1 and the core histone protein, resulting in chromatin decondensation, which is enhanced by myeloperoxidase (MPO). A recent proteome analysis showed that the main components of NETs are DNA, elastase and histones H1, H2A, H2B, H3, and H4 (8); and other components including: neutrophil elastase (NE), MPO, bactericidal/permeability-increasing protein, cathepsin G, lactoferrin, matrix metalloproteinase-9, peptidoglycan recognition proteins, pentraxin, and LL-37.

### SUMMARY OF THE INVENTION

Indeed, the inventors described methods using NET biomarkers as diagnostic biomarkers for inflammatory diseases. COVID-19, Lupus or mCRC are used here as illustrative models for investigating an inflammatory disease. As above indicated, there are numerous biomarkers for NETs formation. Examples in highlighting variation of the respective correlation of NET biomarkers in this invention rely on the determination of the NET main constituents: (i), DNA as determined by examining the amount of circulating DNA (cirDNA) that corresponds to the amount of NET as being degradation by-products that are released into the circulation; (ii) NE; and (iii), MPO; as well as the detection of a blood compound being indirectly associated to NET formation like the anti-cardiolipin auto-antibody. The invention provides threshold values of NE, MPO, cir-nDNA, and cir-mtDNA blood concentrations and of MNR that can be combined to diagnose/screen individuals.

Thus, the invention relates to a method for diagnosing a subject for an inflammatory disease comprising the steps of i) determining in a sample obtained from the subject the level of at least one marker selected in the group consisting in NET protein markers, cir-nDNA, cir-mtDNA and/or a cir-DNA fragmentation index.

Particularly, the invention is defined by its claims.

### DETAILED DESCRIPTION OF THE INVENTION

A first aspect of the invention relates to a method for diagnosing a subject for an inflammatory disease comprising the steps of i) determining in a sample obtained from the subject the level of at least one marker selected in the group consisting in NET protein markers, cir-nDNA, cir-mtDNA and/or a cir-DNA fragmentation index ii) comparing said level determined at step i) with their predetermined reference value and iii) providing that the subject has an inflammatory disease when the level of the NET protein markers, cir-nDNA or cir-DNA fragmentation index determined at step i) is higher than its predetermined reference value or when the level of cir-mtDNA determined at step i) is lower than its predetermined reference value, and providing that the subject has not an inflammatory disease when the level of the NET protein markers, cir-nDNA or cir-nDNA fragmentation index determined at step i) is lower than its predetermined reference value or when the level of cir-mtDNA determined at step i) is higher than its predetermined reference value.

In a particular embodiment, the invention relates to a method for diagnosing a subject for an inflammatory disease comprising the steps of i) determining in a sample obtained from the subject the level of at least two marker selected in the group consisting in NET protein markers, cir-nDNA, cir-mtDNA and/or a cir-DNA fragmentation index ii) comparing said level determined at step i) with their predetermined reference value and iii) providing that the subject has an inflammatory disease when the level of the NET protein markers, cir-nDNA or cir-DNA fragmentation index determined at step i) is higher than its predetermined reference value or when the level of cir-mtDNA determined at step i) is lower than its predetermined reference value, and providing that the subject has not an inflammatory disease when the level of the NET protein markers, cir-nDNA or cir-nDNA fragmentation index determined at step i) is lower than its predetermined reference value or when the level of cir-mtDNA determined at step i) is higher than its predetermined reference value.

In a particular embodiment, the invention relates to a method for diagnosing a subject for an inflammatory disease comprising the steps of i) determining in a sample obtained from the subject the level of at least three marker selected in the group consisting in NET protein markers, cir-nDNA, cir-mtDNA and/or a cir-DNA fragmentation index ii) comparing said level determined at step i) with their predetermined reference value and iii) providing that the subject has an inflammatory disease when the level of the NET protein markers, cir-nDNA or cir-DNA fragmentation index determined at step i) is higher than its predetermined reference value or when the level of cir-mtDNA determined at step i) is lower than its predetermined reference value, and providing that the subject has not an inflammatory disease when the level of the NET protein markers, cir-nDNA or cir-nDNA fragmentation index determined at step i) is lower than its predetermined reference value or when the level of cir-mtDNA determined at step i) is higher than its predetermined reference value.

In a particular embodiment, the invention relates to a method for diagnosing a subject for an inflammatory disease comprising the steps of i) determining in a sample obtained from the subject the level of at least four marker selected in the group consisting in NET protein markers, cir-nDNA, cir-mtDNA and/or a cir-DNA fragmentation index ii) comparing said level determined at step i) with their predetermined reference value and iii) providing that the subject has an inflammatory disease when the level of the NET protein markers, cir-nDNA or cir-DNA fragmentation index determined at step i) is higher than its predetermined reference value or when the level of cir-mtDNA determined at step i) is lower than its predetermined reference value, and providing that the subject has not an inflammatory disease when the level of the NET protein markers, cir-nDNA or cir-nDNA fragmentation index determined at step i) is lower than its predetermined reference value or when the level of cir-mtDNA determined at step i) is higher than its predetermined reference value.

According to the invention, an inflammatory disease can be considered as a NET derived inflammatory process or disease.

Thus, the invention also relates to a method for diagnosing a subject for a NET derived inflammatory process or disease comprising the steps of i) determining in a sample obtained from the subject the level of at least one marker selected in the group consisting in NET protein markers, cir-nDNA, cir-mtDNA and/or a cir-DNA fragmentation index ii) comparing said level determined at step i) with their predetermined reference value and iii) providing that the subject has a NET derived inflammatory process or disease when the level of the NET protein markers, cir-nDNA or cir-DNA fragmentation index determined at step i) is higher than its predetermined reference value or when the level of cir-mtDNA determined at step i) is lower than its predetermined reference value, and providing that the subject has not a NET derived inflammatory process or disease when the level of the NET protein markers, cir-nDNA or cir-nDNA fragmentation index determined at step i) is lower than its predetermined reference value or when the level of cir-mtDNA determined at step i) is higher than its predetermined reference value.

In a particular embodiment, the level of NET protein markers and cir-nDNA or NET protein markers and cir-mtDNA or cir-nDNA and cir-mtDNA are determined in the same blood sample or within the same time frame (<1 month).

In a particular embodiment, the levels of the different markers are combined.

Indeed, the inventor shows that the combined determination of NET protein markers and cir-nDNA or NET protein markers and cir-mtDNA or cir-nDNA and cir-mtDNA allow to obtain a better discrimination between patient with or without an inflammatory disease or a NET derived inflammatory process or disease (see for example the example 11 and 18).

According to the invention, the level of the elastase (NE) can be higher than 2-fold as compared to reference value.

According to the invention, the level of the myeloperoxidase (MPO) can be higher than 2-fold as compared to reference value.

According to the invention, the level of Cir-nDNA can be higher than 2-fold, preferentially 5-fold, as compared to reference value.

According to the invention, the level of Cir-mtDNA can be lower than 2-fold, as compared to reference value.

According to the invention, the level of aCL can be higher by 1.5-fold, as compared to reference value.

In a particular embodiment, the level of the elastase (NE), the myeloperoxidase (MPO) and Cir-nDNA are determined at the same time.

In a particular embodiment, the level of the elastase (NE), the myeloperoxidase (MPO) and Cir-mtDNA are determined at the same time.

In a particular embodiment, the level of the elastase (NE), the myeloperoxidase (MPO) and the MNR are determined at the same time.

In a particular embodiment, the level of the elastase (NE), the myeloperoxidase (MPO), Cir-nDNA and aCL are determined at the same time.

As used herein, the term “combined” denotes that the level of the different markers of the invention (notably NE, MPO, cir-nDNA, cir-mtDNA and MNR) can be combined from a same sample and combined to improve the diagnostic method. In other word, level of one or several markers (NE and/or MPO and/or cir-nDNA and/or cir-mtDNA and/or MNR) can be combined to obtain an improved result. For example, the level of two, three, four or five markers can be combined to obtain an improved result. Particularly, the markers NE, MPO, cir-nDNA and MNR can be combined.

If for example two markers are combined for example, the fact that the levels of the two markers are superior or inferior of their predetermined reference value (depending on the markers) will allow a better result and thus a better diagnostic. In other words, if the significance is obtained for the two markers, the result will be better and will allow a better diagnostic.

The inventors show that the combination of at least two markers or three or four markers allow a better diagnostic of an inflammatory disease. For example, and as shown in the example 18, the combination of NE, MPO, cir-nDNA and MNR prevent the apparition of false positive.

In a particular embodiment, the level of the elastase (NE) can be higher than 2-fold as compared to reference value and the level of cir-nDNA can be higher than 2-fold, preferentially 5-fold, as compared to reference value.

In a particular embodiment, the level of the MPO can be higher than 2-fold as compared to reference value and the level of cir-nDNA can be higher than 2-fold, preferentially 5-fold, as compared to reference value.

In a particular embodiment, the level of the elastase (NE) can be higher than 2-fold as compared to reference value and the level of cir-nDNA can be lower than 2-fold, as compared to reference value.

In a particular embodiment, the level of the MPO can be higher than 2-fold as compared to reference value and the level of cir-nDNA can be lower than 2-fold, as compared to reference value.

In a particular embodiment, the method for diagnosing a subject for an inflammatory disease or the method for diagnosing a subject for a NET derived inflammatory process or disease comprises the determination in a sample obtained from the subject of the level of a NET protein marker with cir-nDNA or a NET protein marker with cir-mtDNA or a NET protein marker with a cir-DNA fragmentation.

According to the invention, the NET protein marker can be MPO, NE, anti-cardiolipin (aCL) and anti-phosphatidylserine.

According to the invention, the cir-mtDNA can be cir-exMT (as defined below).

In another particular embodiment, the method for diagnosing a subject for an inflammatory disease or the method for diagnosing a subject for a NET derived inflammatory process or disease comprises the determination in a sample obtained from the subject of the level of a NET protein marker MPO and NE.

In another particular embodiment, the method for diagnosing a subject for an inflammatory disease or the method for diagnosing a subject for a NET derived inflammatory process or disease comprises the determination in a sample obtained from the subject of the level of cir-nDNA with cir-mtDNA.

In another particular embodiment, the method for diagnosing a subject for an inflammatory disease or the method for diagnosing a subject for a NET derived inflammatory process or disease comprises the determination in a sample obtained from the subject of the level of a NET protein marker with cir-nDNA and/or with cir-mtDNA.

In another particular embodiment, the method for diagnosing a subject for an inflammatory disease or the method for diagnosing a subject for a NET derived inflammatory process or disease comprises the determination in a sample obtained from the subject of the level of a NET protein marker with cir-nDNA and/or with cir-mtDNA and/or the MNR.

In another particular embodiment, the method for diagnosing a subject for an inflammatory disease or the method for diagnosing a subject for a NET derived inflammatory process or disease comprises the determination in a sample obtained from the subject of the level of a MPO with cir-nDNA and/or with cir-mtDNA.

In another particular embodiment, the method for diagnosing a subject for an inflammatory disease or the method for diagnosing a subject for a NET derived inflammatory process or disease comprises the determination in a sample obtained from the subject of the level of a MPO with cir-nDNA and/or with cir-mtDNA and/or the MNR.

In another particular embodiment, the method for diagnosing a subject for an inflammatory discase or the method for diagnosing a subject for a NET derived inflammatory process or disease comprises the determination in a sample obtained from the subject of the level of a NE with cir-nDNA and/or with cir-mtDNA.

In another particular embodiment, the method for diagnosing a subject for an inflammatory disease or the method for diagnosing a subject for a NET derived inflammatory process or disease comprises the determination in a sample obtained from the subject of the level of a NE with cir-nDNA and/or with cir-mtDNA and/or the MNR.

In another particular embodiment, the method for diagnosing a subject for an inflammatory disease or the method for diagnosing a subject for a NET derived inflammatory process or disease comprises the determination in a sample obtained from the subject of the level of the anti-cardiolipin with cir-nDNA and/or with cir-mtDNA.

In another particular embodiment, the method for diagnosing a subject for an inflammatory disease or the method for diagnosing a subject for a NET derived inflammatory process or disease comprises the determination in a sample obtained from the subject of the level of the anti-phosphatidylserine with cir-nDNA and/or with cir-mtDNA.

The different combination described above comprise NET proteins and correspond notably to MPO, NE, anti-cardiolipin and anti-phosphatidylserine.

As used herein, the term “diagnosing a subject for an inflammatory disease” also means “screening a subject for an inflammatory disease”.

### NET Protein Markers

The inventors show that the positive correlation of NET proteins markers such as NE or MPO is specific to COVID-19, cancer and lupus patients and can be considered as a biomarker of these pathologies (which are inflammatory diseases).

As used herein, the term “NET protein markers” denotes protein in the neutrophil extracellular traps like MPO (myeloperoxidase) and NE (neutrophil elastase). NET protein markers can be the myeloperoxidase/DNA complex, the clastase/DNA complex, myeloperoxidase, clastase, citrullinated histones, proteinase 3, cathepsin, lactoferrin, or gelatinase. Indirect associated NET protein markers like anti-phospholipid (anti-cardiolipin (aCL) and, anti-phosphatidylserine) can also be used and are considered as NET protein markers.

As used herein, the term “NET” for “Neutrophil Extracellular Traps” denotes networks of extracellular fibers, primarily composed of DNA from neutrophils, which bind pathogens. Neutrophils are the immune system's first line of defense against infection and have conventionally been thought to kill invading pathogens through two strategies: engulfment of microbes and secretion of anti-microbials. In 2004, a third function was identified: formation of NETs. NETs allow neutrophils to kill extracellular pathogens while minimizing damage to the host cells. Upon in vitro activation with the exogenous pharmacological agent phorbol myristate acetate (PMA), Interleukin 8 (IL-8) or lipopolysaccharide (LPS), neutrophils release granule proteins and chromatin to form an extracellular DNA fibril matrix known as NET through an active process.

According to the auto-antibodies (anti-Phospholipid like anti-cardiolipin and anti-phosphatidylserine), the inventors observed in a great part of mCRC and COVID-19 patients (but not in healthy individuals) have an association of autoantibodies anti-cardiolipin (aCL) with circulating NETs markers. ACL is part of the anti-phospholipid antibody family (aPL) characteristic of the anti-phospholipid syndrome (APS, 36) an immune-mediated disorder resulting in pregnancy morbidity and arterial or venous thrombotic events. While aPL is present in 1-5% of the general population, APS prevalence is 40-50/100,000 subjects (Rato). However, this prevalence can increase to 50% among elderly patients with chronic diseases ( . . . ). It should also be noted that exacerbated NET formation has been linked to anti-phospholipid syndrome (APS) in numerous auto-immune and non-auto-immune pathologies, such as lupus that showing elevated level of aCL (5).

In respect to cancer, several works report higher aPL levels in various haematological and solid tumors (36), with aPL positive cancer patients varying from 5% to 70% (36). Since the risk of thrombosis is 4-60-fold higher in cancer patients than in the general population, it has been suggested that an elevated level of aPL might trigger thrombosis in cancer patients. A meta-analysis revealed that patients with gastrointestinal, genitourinary and lung cancer are at a higher risk of developing aPL (35). While aPL such as aCL appears as an essential step towards preventing the occurrence of thrombosis in cancer patients, the direct or indirect implication of aPL in the thrombophilic process remains unclear. It is clear, however, that neutrophils and NETs contribute to APS pathophysiology (35). Assuming that cirDNA are indirect NET markers, and are a marker of tumor mass in mCRC, our study suggests the association of an increase of circulating NETs markers and disease severity. Unusually, there is a high prevalence of thromboembolic events in COVID-19 patients ( . . . ). The inventors may speculate that cirDNA and NETs by-products are markers of the inflammation associated with solid tumors, and more globally with inflammatory diseases. It is possible, therefore, that cirDNA concentrations associated with other NETs circulating markers could provide significant information about cancer severity, cancer prognosis or treatment guidance, which could constitute a significant advance in cancer along with the advent of immunotherapy and the growing knowledge of tumor immunology. Basically, the high association of anti-phospholipid (anti-cardiolipin) levels with NE, MPO, and cirDNA plasma concentration suggested that NETosis might be a critical factor in the immunological response/phenomena linked to tumor progression.

As illustrated in example 5, most of COVID-19 and mCRC patient plasma contain ACL, supporting the notion that inflammatory diseases are associated with NET formation and generation of auto-antibodies. Given the auto-stimulatory nature of the interplay between neutrophil stimulation/NET exacerbation/auto-antibody production, ACL presence and level can be controlled once inflammatory disease is diagnosed, for instance even disappearance of disease symptoms such as in long COVID-19.

### cir-DNA and cir-DNA Fragmentation Index

The inventors show that the positive correlation of cir-DNA and cir-DNA fragmentation index level are specific to COVID-19, cancer and lupus patients and can be considered as a biomarker of these pathologies and can be used as example of inflammatory diseases.

According to the invention, the term “cir-DNA” denotes also circulating cell-free DNA (ccfDNA) released by cells or mitochondria.

According to the invention and as used, the term cir-DNA denotes cir-nDNA (nuclear DNA) and cir-mtDNA (mitochondrial DNA) fragments.

According to the invention, the cir-DNA can be single or double stranded DNA fragment.

As used herein, the term “single stranded DNA fragment” denotes single stranded (compared to double stranded) fragment of DNA which can have different size of nucleic acids.

As used herein, the term “double stranded DNA fragment” denotes double stranded (compared to single stranded) fragment of DNA which can have different size of nucleic acids.

As used herein, the term “cir-nDNA” denotes the circulating nuclear DNA and the term cir-mtDNA denotes the circulating mitochondrial DNA.

According to the invention, the level of the nuclear markers cirDNA (cir-nDNA) and mitochondrial cirDNA (cir-mtDNA) also correspond to the concentration of cell-free nuclear cir-DNA and cell-free mitochondrial cir-DNA.

According to the extracellular mitochondrial DNA (cir-mtDNA), beside necrosis or apoptosis extracellular mitochondrial DNA is mainly produced from the vital NETosis. In vital NETosis the nuclear membrane remains intact despite releasing DNA through NET formation and is able to continue normal neutrophil functioning. Stimuli received by toll-like receptors (TLR) leads to the decondensation of chromatin and vesicle formation altering the nuclear envelope for DNA release. Vital NETosis is of two types, one that does not rely on ROS activity while the other does. Vital NETosis that is ROS dependent results in the expulsion of mitochondrial DNA instead of nuclear DNA in NETs.

The inventors demonstrated that the cir-mtDNA detected from plasma mainly originate from circulating extracellular mitochondria (cir-exMT). They suggested that circulating-mitochondrial DNA is also associated with approximately 18% small extracellular vesicles, 4% exosomes and 1% protein complexes.

As used herein, the term “cir-DNA fragmentation index” denotes some ratio and/or differences between the different cir-DNA fragments (cir-nDNa and cir-mtDNA). These ratio and/differences can be determined between cir-DNA fragment size ranges or between the frequency at a specific size. The cir-DNA fragmentation index denotes for example the DNA Integrity index (DII) that is determined from the ratio of the amount of all or nearly all fragments (for instance all the fragments over 67 bp) over the amount of all the fragment lengths over a 220 bp-305 range, for instance all the fragments over 305 bp.

The inventors calculated the DNA Integrity index (DII) (see the table 1 in the examples). DII is determined as the ratio of long over short fragments of cir-nDNA, by analyzing by Q-PCR the quantity of amplicons generated by targeting here a 67 bp and 305 bp sequence in the monogenic KRAS gene.

Thus, the invention also relates to a method for diagnosing a subject for an inflammatory disease comprising the steps of i) determining in a sample obtained from the subject the level of long and short fragments of cir-nDNA, ii) calculated the ratio of long over short fragments of cir-nDNA (DII), iii) comparing said level determined at step i) with their predetermined reference value and iv) providing that the subject has an inflammatory disease when the calculated ratio determined at step ii) is higher than its predetermined reference value, and providing that the subject has not an inflammatory disease when the calculated ratio determined at step i) is lower than its predetermined reference value.

According to the invention, quantifying the level of long and short fragments of cir-DNA (cir-nDNA and cir-mtDNA) is calculated with fragments between 67 bp and 305 bp.

Particularly, long fragments are higher than 200 pb and more particularly higher than 260 bp.

Particularly, short fragments are lower than 150 pb and more particularly lower than 80 bp.

In one embodiment, the cir-DNA fragments are derived from the monogenic KRAS gene.

The inventors also made ratios using the biomarkers of the invention to distinguish a subject hospitalized for an inflammatory disease (=non-severe) versus a subject hospitalized for an inflammatory disease in an ICU (Intensive Care Unit) (=severe) with healthy subject. Note that in the case of a severe inflammatory disease, the patients will need for example oxygen. For example, the inventors used the ratio of the cir-mtDNA content over cir-nDNA content (called here as MNR).

Thus, in one embodiment, the invention relates to a method for diagnosing a subject for an inflammatory disease comprising the steps of i) determining in a sample obtained from the subject the level of cir-mtDNA and cir-nDNA ii) calculating the MNR ratio (cir-mtDNA over cir-nDNA ratio), iii) comparing said ratio determined at step ii) with a predetermined reference value and iv) providing that the subject has an inflammatory disease when the calculated ratio determined at step ii) is lower than its predetermined reference value and providing that the subject has not an inflammatory disease when the calculated ratio determined at step ii) is higher than its predetermined reference value.

According to the invention, the level of the MNR can be lower than 3-fold, as compared to reference value.

As used herein the term “nucleic acid” has its general meaning in the art and refers to refers to a coding or non-coding nucleic sequence. Nucleic acids include DNA (deoxyribonucleic acid) and RNA (ribonucleic acid) nucleic acids. Example of nucleic acid thus include but are not limited to DNA, mRNA, tRNA, rRNA, tmRNA, miRNA, piRNA, snoRNA, and snRNA. Nucleic acids thus encompass coding and non-coding region of a genome (i.e. nuclear or mitochondrial).

As used herein, the term “nuclear nucleic acid” has its general meaning in the art and refers to a nucleic acid originating from the nucleus of cell. The term nuclear nucleic acid encompasses all forms of the nucleic acids excepting those originating from the mitochondria. The term nuclear nucleic acid is thus defined in opposition to the term “mitochondrial nucleic acid”. Mitochondria are indeed structures within cells that convert the energy from food into a form that cells can use. Although most DNA is packaged in chromosomes within the nucleus, mitochondria also have a small amount of their own DNA. This genetic material is known as “mitochondrial DNA” or “mtDNA”. In humans, mitochondrial DNA spans about 16,500 DNA building blocks (base pairs), representing a small fraction of the total DNA in cells. Mitochondrial DNA contains 37 genes, all of which are essential for normal mitochondrial function: ATP6; ATP8; COX1; COX2; COX3; CYTB; ND1; ND2; ND3; ND4; ND4L; ND5; ND6; RNR1, RNR2 TRNA; TRNA; TRNC; TRND; TRNE; TRNF; TRNG; TRNI; TRNK; TRNL1; TRNL2; TRNM; TRNN; TRNN; TRNP; TRNQ; TRNR; TRNS1; TRNS2; TRNT; TRNV; TRNW; and TRNY. Genes encoding for NADH dehydrogenase (complex I) include MT-ND1, MT-ND2, MT-ND3, MT-ND4, MT-ND4L, MT-ND5, and MT-ND6. Genes encoding for Coenzyme Q-cytochrome c reductase/Cytochrome b (complex III) include MT-CYB. Gene encoding for cytochrome c oxidase (complex IV) include MT-CO1, MT-CO2, MT-CO3. Gene enconding for ATP synthase (complex V) include MT-ATP6, and MT-ATP8. Gene encoding for humanin include MT-RNR2 (encoding both ribosomal 16S and humanin). MT-RNR1 and MT-RNR2 genes providing instruction to produce ribosomal 12S and 16S respectively. The 22 species of mitochondrial tRNAs (mt tRNAs) encoded by mtDNA involved in mitochondrial protein synthesis machinery. Human mitochondrial DNA (mtDNA) has three promoters, H1, H2, and L (heavy strand 1, heavy strand 2, and light strand promoters). Mitochondrial genome also comprises control regions or d-loop sequences. Mitochondrial nuclear acids are known per se by the skilled person (e.g. NCBI Reference Sequence: NC_012920.1). Thirteen of these genes provide instructions for making enzymes involved in oxidative phosphorylation. Oxidative phosphorylation is a process that uses oxygen and simple sugars to create adenosine triphosphate (ATP), the cell's main energy source. The remaining genes provide instructions for making molecules called transfer RNA (tRNA) and ribosomal RNA (rRNA), which are chemical cousins of DNA. These types of RNA help assemble protein building blocks (amino acids) into functioning proteins.

By “cell-free nucleic acid” or “cfDNA” it is meant that the nucleic acid is released by the cell and present in the sample. In some embodiments, the cell-free nucleic acid is circulating cell-free DNA (ccfDNA) and it is casy and routine for one of ordinary skill in the art to distinguish mitochondrial ccf nucleic acids” or “mitochondrial ccfDNA” from “nuclear ccfDNA”. Actually, mitochondrial ccfDNA encompasses any DNA mitochondrial nucleic acid and in opposition nuclear ccfDNA encompasses any DNA nuclear nucleic acid.

According to the invention, to amplify the DNA fragments, primers can be used (for PCR for example).

As used herein, the term “primer” refers to an oligobp, whether occurring naturally as in a purified restriction digest or produced synthetically, which is capable of acting as a point of initiation of nucleic acid sequence synthesis when placed under conditions in which synthesis of a primer extension product which is complementary to a nucleic acid strand is induced, i.e. in the presence of different bp triphosphates and a polymerase in an appropriate buffer (“buffer” includes pH, ionic strength, cofactors etc.) and at a suitable temperature. Typically, a primer has a length of 10; 11; 12; 13; 14; 15; 16; 17; 18; 19; 20; 21; 22; 23; 24; 25; 26; 27; 28; 29; or 30 bp. One or more of the bp of the primer can be modified for instance by addition of a methyl group, a biotin or digoxigenin moicty, a fluorescent tag or by using radioactive bp. A primer sequence need not reflect the exact sequence of the template. For example, a non-complementary bp fragment may be attached to the 5′ end of the primer, with the remainder of the primer sequence being substantially complementary to the strand. Primers are typically labelled with a detectable molecule or substance, such as a fluorescent molecule, a radioactive molecule or any others labels known in the art. Labels are known in the art that generally provide (either directly or indirectly) a signal. The term “labelled” is intended to encompass direct labelling of the probe and primers by coupling (i.e., physically linking) a detectable substance as well as indirect labeling by reactivity with another reagent that is directly labeled. Examples of detectable substances include but are not limited to radioactive agents or a fluorophore (e.g. fluorescein isothiocyanate (FITC) or phycoerythrin (PE) or Indocyanine (Cy5)).

Note, low-pass Whole Genome Sequencing enables the observation of the precise fragment size profile up to ˜1000 bp (37) and be a useful tool to observe variation of cirDNA fragment size profile.

### Others Use of cir-DNA

The inventors show that the different cir-DNA (cir-nDNA or cir-mtDNA) can be use in different way to diagnose/screen an inflammatory disease.

For example, the cir-DNA can be use as single or double stranded DNA fragment. Ratio between single or double stranded DNA fragments can be made, determination of the level of different length of fragment or fragment length ranges can also be made.

Thus the invention also relates to a method for diagnosing a subject for an inflammatory disease comprising the steps of:
i. extracting the cir-DNA (cir-nDNA or cir-mtDNA) from a sample obtained from the subject;
ii. determining the level of at least one single or double stranded DNA fragment having a length between 20 to 440 base pairs (bp);
iii. comparing the level determined at step ii) with a predetermined reference value and;
iv. concluding that the subject suffers from an inflammatory disease when the level determined at step iii) differ from the predetermined reference value.

The invention also relates to a method for diagnosing a subject for an inflammatory disease comprises the steps of:
i. extracting the cir-DNA (cir-nDNA or cir-mtDNA) from a sample obtained from the subject;
ii. determining the level of a first single or double stranded DNA fragment having a length between 20 to 440 bp;
iii. determining the level of a second single or double stranded DNA fragment having a length between 20 to 440 bp;
iv. calculating the ratio of the level determined at step ii) to the level determined at step iii) or alternatively the ratio of the level determined at step iii) to the level determined at step ii;
V. comparing the ratio determined at step iv) with a predetermined corresponding reference value and;
vi. concluding that the subject suffers from an inflammatory disease when the ratio determined at step iv) differ from the predetermined corresponding reference value.

Cir-DNA fragment profile can be determined precisely by LP-WGS. As shown in FIG. 10, cancer cir-DNA size profile shows a slight but reliable shift to the lower size a peak at 167-168 bp. Several parameters as determined from the size profile enable to characterize mCRC and COVID-19 patients and enable the screening of cancer patients.

Note, SLE plasma cir-nDNA size profile appears to vary as compared to that of healthy individuals. The proportion of the fragments of range 30-90, 90-168 and 300-420 are slightly higher, lower and higher than that of healthy individuals (FIG. 16).

According to the previous methods the single or double stranded DNA fragments for which the level (or the concentration) is determined can have a length of less than 90 bp or more than 167 bp or between 90 to 167 bp or between 142 to 152 or between 167 to 220 or between 220 to 440 bp. For the ratio the single or double stranded DNA fragments can have a length of 40 bp, 90 bp, 145 bp, 167 bp or 320 bp.

### Use of the Different Markers of the Invention

The inventors also combined the markers of the invention to improve the power of the methods of the invention.

In another aspect, the invention also relates to a method for diagnosing a subject for an inflammatory disease comprising the steps of i) determining in a sample obtained from the subject the level of at least one marker selected in the group consisting in NET protein markers, cir-nDNA and cir-mtDNA ii) calculating a ratio with at least one of said marker, iii) comparing said ratio determined at step ii) with a predetermined reference value and iv) providing that the subject has an inflammatory disease when the calculated ratio determined at step ii) is lower than its predetermined reference value and providing that the subject has not an inflammatory disease when the calculated ratio determined at step ii) is higher than its predetermined reference value.

According to the following method, the ratio is calculated with two markers selected in the group consisting in NET protein markers, cir-nDNA and cir-mtDNA.

In a particular embodiment, the method is suitable to distinguishing a healthy subject with a subject hospitalized for an inflammatory disease and with a subject hospitalized for an inflammatory disease in a ICU.

In a particular embodiment, the particular calculated ratio can be: NE/Cir-mtDNA, MPO/cir-mtDNA and NE x MPO/Cir-mtDNA.

### Diseases of the Invention

According to the invention, the inflammatory disease can be a pathogen infection, an autoimmune disease like lupus (SLE) or a cancer.

According to the invention, the inflammatory disease can be a pathogen infection and particularly a pathogen respiratory infection.

A pathogen respiratory infection can be a pathogen lung infection.

As used herein, the term “a pathogen lung infection” denotes a lung infection induced by a biological pathogen or in other word an infectious agent.

According to the invention, the pathogen can be a virus, bacterium, protozoan, prion, viroid, or fungus.

According to the invention, the bacterium can be selected from the group consisting of: Streptococcus pneumoniae; Staphylococcus aureus; Haemophilus influenza, Myoplasma species, Moraxella catarrhalis, Escherichia, e.g., E. coli, Enterobacter, Erwinia, Klebsiella, Proteus, Salmonella, e.g., Salmonella enterica serovar, Typhimurium, Serratia, e.g., Serratia marcescans, and Shigella, as well as Bacilli such as B. subtilis and B. licheniformis, Pseudomonas such as P. aeruginosa, Campylobacter, Mycobacterium tuberculosis, and Streptomyce.

According to the invention, the fungus can be selected from the group consisting of: aspergillus, Candida albicans and Cryptococcus neoformans.

In another particular embodiment, the pathogen lung infection is induced by a respiratory virus.

Particularly, the respiratory virus can be Influenza virus, such as the Influenza A virus (IAV) or the Influenza B virus (IAB), adenovirus, metapneumovirus, cytomegalovirus, 25 parainfluenza virus (e.g., hPIV-1, hPIV-2, hPIV-3, hPIV-4), the human rhinovirus (HRV), the Human respiratory syncytial virus (HRSV) or a coronavirus.

As used herein, the term “coronavirus” has its general meaning in the art and refers to any member of members of the Coronaviridae family. Coronavirus is a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. In particular, coronavirus RNAs encode: (1) an RNA-dependent RNA polymerase; (2) N-protein; (3) three envelope glycoproteins; plus (4) three non-structural proteins. In particular, the coronavirus particle comprises at least the four canonical structural proteins E (envelope protein), M (membrane-protein), N (nucleocapsid protein), and S (spike protein). The S protein is cleaved into 3 chains: Spike protein S1, Spike protein S2 and Spike protein S2′. Production of the replicase proteins is initiated by the translation of ORFla and ORFlab via a −1 ribosomal frame-shifting mechanism. This mechanism produces two large viral polyproteins, ppla and pplab, that are further processed by two virally encoded cysteine proteases, the papain-like protease (PLpro) and a 3C-like protease (3CLpro), which is sometimes referred to as main protease (Mpro). Coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric coV (ATCC accession #VR-1475), human coV 229E (ATCC accession #VR-740), human coV OC43 (ATCC accession #VR-920), Middle East respiratory syndrome-related coronavirus (MERS-Cov) and SARS-coronavirus (Center for Disease Control), in particular SARS-Cov1 and SARS-Cov2.

According to the invention, the coronavirus can be a MERS-CoV, SARS-CoV, SARS CoV-2 or any new future family members.

As used herein, the SARS CoV-2 is responsible of the Covid-19 disease.

Particularly, the inventors showed in the example 17, that the markers of the invention can be used to diagnosis (detect) patient with a long-Covid (long Covid-19) which means patients with still “experienced” symptoms of the Covid-19 6 months or more after the post-acute infection.

Thus, in particular embodiment, the Covid-19 disease is a long Covid-19 disease.

As used herein, the term “long Covid-19” denotes that a condition characterized by long-term consequences persisting or appearing after the typical convalescence period of COVID-19. It is also known as post-COVID-19 syndrome, post-COVID-19 condition, post-acute sequelae of COVID-19 (PASC), or chronic COVID syndrome (CCS). Long COVID can affect nearly every organ system, with sequelae including respiratory system disorders, nervous system and neurocognitive disorders, mental health disorders, metabolic disorders, cardiovascular disorders, gastrointestinal disorders, musculoskeletal pain, and anemia.[6] A wide range of symptoms are commonly reported, including fatigue, malaise, headaches, shortness of breath, anosmia (loss of smell), parosmia (distorted smell), muscle weakness, low fever and cognitive dysfunction.

According to the invention, the inflammatory disease can be a cancer.

According to the invention, the cancer may be selected in the group consisting of adrenal cortical cancer, anal cancer, bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

In a particular embodiment, the cancer is a metastatic cancer.

In another particular embodiment, the cancer is a colorectal cancer or a metastatic colorectal cancer (mCRC).

### Cancer as an Example of a Inflammatory Disease

The inventors demonstrated that concomitant analyses of NETs markers (NE, MPO and cirDNA) enables the differentiation of mCRC patients from healthy individuals. The study of the inventors is the first to show the close association of NET markers, cirDNA and aPL in cancer patients, suggesting that the examination of these markers might be useful in preventing the occurrence of thrombosis in cancer patients. Lastly, our observations contribute to the understanding of the imbalance which cancer can cause between the immunological system and hemostasis. This deepened understanding may prove useful improving long term cancer survival rates.

According to the invention, the inflammatory disease can be an autoimmune disease.

The autoimmune disease may be selected in the group consisting in celiac disease, diabetes mellitus type 1, Graves' disease, inflammatory bowel disease, multiple sclerosis, psoriasis, asthma, obesity, fatty liver diseases, endometriosis, rheumatoid arthritis, and systemic lupus erythematosus (SLE).

According to the invention, inflammatory disease can also be Alzheimer's, and Parkison's disaeses or Vasculitis,

### Others Definitions of the Invention

As used herein the term “sample” refers to any biological sample obtained from the subject that is liable to contain cell-free nucleic acids. Typically, samples include but are not limited to body fluid samples, such as blood, ascite, urine, amniotic fluid, feces, saliva or cerebrospinal fluids. In some embodiments, the sample is a blood sample. By “blood sample” it is meant a volume of whole blood or fraction thereof, e.g., serum, plasma, etc. Any methods well known in the art may be used by the skilled artisan in the art for extracting the free cell nucleic acid from the prepared sample. For example, the method described in the EXAMPLE may be used.

As used herein, the term “subject” denotes a mammal. Typically, a subject according to the invention refers to any subject (preferably human) afflicted with an inflammatory disease like Covid-19 disease. The term “subject” also refers to a subject with no disease.

### Methods Used to Determine the Levels of the Biomarkers of the Invention

Methods to determine the levels of cir-DNA (and cir-DNA fragment) may be accomplished by any method, including without limitation chromatography, direct sequencing, spectrometry or Q-PCR.

Direct sequencing may be accomplished by any method, including without limitation chemical sequencing using the Maxam-Gilbert method, spectrometry and particularly mass spectrometry sequencing and sequencing using a chip-based technology.

In the chemical sequencing, base specific modifications result in a base specific cleavage of the radioactive or fluorescently labeled DNA fragment. With the four separate base specific cleavage reactions, four sets of nested fragments are produced which are separated according to length by polyacrylamide gel electrophoresis (PAGE). After autoradiography, the sequence can be read directly since each band (fragment) in the gel originates from a base specific cleavage event. Thus, the fragment lengths in the four “ladders” directly translate into a specific position in the DNA sequence.

In the enzymatic sequencing, the four base specific sets of DNA fragments are formed by starting with a primer/template system elongating the primer into the unknown DNA sequence area and thereby copying the template and synthesizing a complementary strand by DNA polymerases, such as Klenow fragment of E. coli DNA polymerase I, a DNA polymerase from Therm us aquaticus, Taq DNA polymerase, or a modified T7 DNA polymerase, Sequenase, in the presence of chain-terminating reagents.

Several new methods for DNA sequencing (High-throughput sequencing (HTS) methods) were developed in the mid to late 1990s and were implemented in commercial DNA sequencers by the year 2000. Together these were called the “next-generation” or “second-generation” sequencing methods. These HTS included but are not limited to: Single-molecule real-time sequencing, Ion semiconductor, Pyrosequencing, Sequencing by synthesis, Sequencing by ligation, Nanopore Sequencing, Chain termination and Sequencing by hybridization. Some of these methods allow a Whole Gene Sequencing (WGS), Whole Exome Sequencing (WES) or a Targeted Sequencing.

Methods to determine the levels of proteins (like Ne or MPO) or antibody (like anti-phospholipid (anti-cardiolipin and, anti-phosphatidylserine)) may be accomplished by any method, including without limitation ELISA.

Typically protein or antibody concentration may be measured for example by capillary electrophoresis-mass spectroscopy technique (CE-MS) or ELISA performed on the sample.

Such methods comprise contacting a sample with a binding partner capable of selectively interacting with proteins present in the sample. The binding partner is generally an antibody that may be polyclonal or monoclonal, preferably monoclonal.

The presence of the protein or antibody can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, capillary electrophoresis-mass spectroscopy technique (CE-MS).etc. The reactions generally include revealing labels such as fluorescent, chemioluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

In a particular embodiment, the detection of the level of the proteins or antibodies of the invention can be performed by flow cytometry.

### Predetermined Corresponding Reference Values

Typically, the predetermined corresponding reference value can be relative to a number or value derived from population studies, including without limitation, subjects of the same or similar age range, subjects in the same or similar ethnic group, subjects at risk of inflammatory disease like Covid-19 disease, subjects having a severe inflammatory disease (hospitalized in ICU) and subject without inflammatory disease (healthy subject). Such predetermined corresponding reference values can be derived from statistical analyses and/or risk prediction data of populations obtained from mathematical algorithms and computed indices of the discasc.

According to the invention, the terms “level”, “concentration” and “quantity” can be used in a equivalent manner.

Notably, the term “level” for cir-nDNA or cir-mtDNA denotes the frequency, proportion, quantity, concentration or number of fragments of cir-nDNA or cir-mtDNA for a done length. For example, the level or a DNA fragment (cir-nDNA or cir-mtDNA) having a length between 20 to 440 bp denotes the frequency or proportion of DNA fragment having a length between 20 to 440 bp.

Particularly, for cir-nDNA or cir-mtDNA the terms number or proportion will be more used.

Typically, the predetermined corresponding reference value is a threshold value or a cut-off value. A “threshold value”, “reference value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the concentration of the markers of the invention (e.g. cir-DNA for example) in properly banked historical subject samples may be used in establishing the predetermined corresponding reference value. In some embodiments, the predetermined corresponding reference value is the median measured in the population of the subjects for the marker of in the invention (e.g. cir-DNA for example). In some embodiments, the threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the concentration of the marker of the invention (e.g. cir-DNA for example) in a group of reference, one can use algorithmic analysis for the statistic treatment of the levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator the reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0,ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined corresponding reference value is typically determined by carrying out a method comprising the steps of:
a) providing a collection of samples from subjects;
b) providing, for each sample provided at step a), information relating to the actual clinical profile of the subject (healthy or suffering from a inflammatory disease like Covid-19 disease);
c) providing a serial of arbitrary quantification values;
d) determining the concentration of the marker of the invention (e.g. cir-DNA for example t) for each sample contained in the collection provided at step a);
e) classifying said blood samples in two groups for one specific arbitrary quantification value provided at step c), respectively: (i) a first group comprising samples that exhibit a quantification value for level that is lower than the said arbitrary quantification value contained in the said serial of quantification values; (ii) a second group comprising samples that exhibit a quantification value for said level that is higher than the said arbitrary quantification value contained in the said serial of quantification values; whereby two groups of samples are obtained for the said specific quantification value, wherein the samples of each group are separately enumerated;
f) calculating the statistical significance between (i) the quantification value obtained at step c) and (ii) the actual clinical profile of the subjects from which samples contained in the first and second groups defined at step f) derive;
g) reiterating steps f) and g) until every arbitrary quantification value provided at step d) is tested;
h) setting the said predetermined corresponding reference value as consisting of the arbitrary quantification value for which the highest statistical significance (most significant) has been calculated at step g).

Thus in some embodiments, the predetermined corresponding reference value thus allows discrimination between healthy subject and subjects suffering from an inflammatory disese. Practically, high statistical significance values (e.g. low P values) are generally obtained for a range of successive arbitrary quantification values, and not only for a single arbitrary quantification value. Thus, in one alternative embodiment of the invention, instead of using a definite predetermined corresponding reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the diagnosis can be determined by comparing the co centration of the marker of the invention (e.g. cir-DNA for example) with the range of values which are identified. In certain embodiments, a cut-off value thus consists of a range of quantification values, e.g. centered on the quantification value for which the highest statistical significance value is found (e.g. generally the minimum p value which is found).

According to the invention, the variation of the concentration of the markers of the inventions (Nuclear cirDNA (cir-nDNA), mitochondrial cirDNA concentration (Cir-mtDNA), MPO (myeloperoxidase) and NE (neutrophil elastase)), MNR and cirDNA fragmentation markers may be evaluated/determined.

According to the invention, discriminations are globally determined either from proportion of a single concentration of the markers of the invention or a concentration group of the markers of the invention, or from a ratio of two or more concentration of the markers of the invention.

As determined by the quantitative blood values of NE, MPO, cir-nDNA and cir-mtDNA these analytes discriminated mCRC, COVID-19, and SLE patients from healthy (EFS) individuals. The observed correlation of these markers supported this postulate. Thus COVID-19, SLE and mCRC show similar characteristics illustrating their common properties as inflammatory diseases, despite their different nature. While some reports previously indicated that NETs and some NET by-products such as NE, MPO may be associated in these disorders, our invention provide not only threshold value to diagnose an individual, but also provide the innovation in combining them. This enables to partially or totally combine them in an artificial intelligence machine learning system allowing the definition of an optimal test for diagnosis and to follow-up individuals with inflammatory diseases. According to the examples shown in this invention, the threshold for NE, MPO, cir-nDNA, cir-mtDNA and MNR are respectively at least, but not limited to 15 ng/ml, 20 ng/ml, 7 ng/mL, 0.1 ng/ml and 0.014, or preferably 21 ng/mL, 21.5 ng/ml, 9 ng/mL, 0.06 ng/ml and 0.01. For instance, an individual showing plasmatic concentration over at least 21 ng/mL, and/or over at least 21.5 ng/ml, and/or over at least 9 ng/ml, and/or under at least 0.06 ng/ml and/or under 0.01, is diagnosed as having an inflammatory disease. This test may serve either as a screening test or a patient follow-up test.

In other words, when the concentration (quantity) of NE is at least superior to 15 ng/ml or at least superior to 21 ng/ml, the subject has an inflammatory disease, when the concentration (quantity) of MPO is at least superior to 20 ng/ml or at least superior to 21.5 ng/mL, the subject has an inflammatory disease, when the concentration (quantity) of cir-nDNA is at least superior to 7 ng/ml or at least superior to 9 ng/ml, the subject has an inflammatory disease, when the concentration (quantity) of cir-mtDNA is at least inferior to 0.1 ng/ml or at least inferior to 0.06 ng/ml, the subject has an inflammatory disease and when the level of MNR is at least inferior to 0.1 or at least inferior to 0.01, the subject has an inflammatory disease. Note that for the MNR, since it is a ratio (cir-mtDNA over cir-nDNA ratio) there is no unit.

Finally, the invention resides in using threshold values either individually or combined: values above the thresholds of NE, MPO, cir-nDNA, and values below the thresholds of cir-mtDNA or MNR are considered to indicate an inflammatory process (or a NET derived inflammatory process), individually/independently or in a combined way.

### Therapeutic Applications

The invention highlights the role of the NETs and auto-antibodies in the pathogenesis of inflammatory process and diseases. The detection of a inflammatory process as determined by one or more of the cited biomarkers according to specific thresholds, point to the therapeutic need of inhibiting NET formation and auto-antibodies generation.

Thus, the methods of the present invention can also be suitable for determining whether a subject is eligible or not to an anti-inflammatory disease treatment. An anti-inflammatory treatment typically consists of nonsteroidal anti-inflammatory drugs (NSAIDs) like aspirin, ibuprofen, and naproxen, antileukotrines, immune selective anti-inflammatory derivatives (ImSAIDs) or DNase-1, anti-IL1 or IL6 treatments.

Anti-inflammatory treatments may be combined with a further therapeutic active agent useful to treat inflammatory disease or the symptoms induced by inflammatory disease. For example, further agent may be selected in the group consisting of bronchodilators like β2 agonists and anticholinergics, corticosteroids, beta2-adrenoceptor agonists like salbutamol, anticholinergic like ipratropium bromide or adrenergic agonists like epinephrine. Further agent may be also an antiviral compound like amantadine, rimantadine or pleconaril.

Anti-inflammatory disease treatment ca also be an antibody against anti-phospholipid.

The invention also relates a method for treating an inflammatory disease diagnosed by the method of the invention comprising administering to a subject in need thereof an anti-inflammatory disease treatment as described above.

### Kit

A further object of the invention relates to kit comprising means for performing the methods of the present invention. Typically, the kit comprises means for detection of the presence or absence of the phenotypic markers of interest.

In some embodiments, the present invention relates to a kit for diagnosing a subject for an inflammatory disease wherein said kit comprises means for determining t marker selected in the group consisting in NET protein markers (like MPO, NE, anti-cardiolipin (aCL) and anti-phosphatidylserine), cir-nDNA or cir-mtDNA.

Typically, the kit described above will also comprise one or more other containers, containing for example, wash reagents, and/or other reagents capable of quantitatively detecting the presence of bound antibodies. The kit also contains agents suitable for performing intracellular flow cytometry such as agents for permeabilization and fixation of cells. Typically, compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the sample, a container which contains the antibody(s) used in the assay, containers which contain wash reagents (such as phosphate buffered saline, Tris-buffers, and like), and containers which contain the detection reagent.

The invention will be further illustrated by the following figures and examples. However, these examples and figures should not be interpreted in any way as limiting the scope of the present invention.

### FIGURES

FIG. 1: Box plot values of NE, MPO, and cir-nDNA in healthy; and hospitalized, ICU, and all COVID-19 patients.

FIG. 2: Box plot values of cir-mtDNA and MNR in healthy; and hospitalized, ICU, and all COVID-19 patients.

FIG. 3: Pearson's correlations of NE, MPO, cir-nDNA, cir-mtDNA and MNR values. A, COVID-19 ICU (N=18); B, COVID-19 hospitalized (N=14); and C, healthy individuals (HI) (113).

FIG. 4: ROC curves analysis comparing COVID-19 patients and healthy individuals when analysing MPO values.

FIG. 5: ROC curves analysis comparing COVID-19 patients and healthy individuals when analysing NE values.

FIG. 6: ROC curves analysis comparing COVID-19 patients and healthy individuals when analysing cir-nDNA values.

FIG. 7: ROC curves analysis comparing COVID-19 patients and healthy individuals when analysing cir-mtDNA values.

FIG. 8: ROC curves analysis comparing COVID-19 patients and healthy individuals when analysing MNR values.

FIG. 9: ROC curves analysis comparing COVID-19 patients and healthy individuals when analysing DII values.

FIG. 10: Size profile as determined by low pass WGS of ICU (A) and Hospitalized (B) patients (full line) as compared to that of healthy individuals (dotted line).

FIG. 11: MPO, NE and Cir-nDNA (RefA) values as determined in mCRC (full bars) and EFS (gray bars). Statistical differences as determined by the calculation of the P values (Table at the bottom of the figure).

FIG. 12: Pearson correlation values when analysing MPO, NE and cir-nDNA.

FIG. 13: ROC curves analysis when discriminating mCRC patients and healthy individuals with using NE, MPO, cir-nDNA, MPO plus NE, and cir-nDNA plus MPO plus NE.

FIG. 14: NE, MPO, cir-nDNA (RefA) and cir-mtDNA (RefM) mean values as determined in SLE patients (black bars) and healthy (EFS) patients (gray bars). Statistical analysis and P values (table at the bottom of the figure.

FIG. 15: Pearson correlation analysis of NE, MPO, DII, cir-nDNA, cir-mtDNA, MNR and DII valuesin the ten SLE patients.

FIG. 16: cir-nDNA size profile from an illustrative SLE plasma sample (L4) as compared to that of the mean of seven healthy plasmas (dotted line).

FIG. 17: NE, MPO, and cir-nDNA (cirDNA) are characterizing inflammatory diseases. Each marker appears to help in discriminating mCRC, COVID-19, and EFS patients from healthy (EFS) individuals, suggesting a high screening power when combining them.

FIG. 18: Auto-anticorps cardiolipin (aCL) appears elevated in COVID-19 patients as compared to healthy subjects. AI, anticorps index. Statistical differences were observed between healthy individuals and each COVID-19 patient groups (hospitalized, ICU, or ICU+hospitalized). Histogram represent the mean +/−SD.

FIG. 19: Pearson correlation values of aCL and the various other inflammatory parameters analysed in this invention. A: ICU COVID-19 patients; B: Hospitalized patients.

FIG. 20: Comparison of anti-cardiolipin (aCL) auto-antibodies mean level in plasma of mCRC patients and healthy individuals. The anti-cardiolipin (aCL) auto-antibodies level was determined by Elisa test and expressed in arbitrary unit (IA, index of auto-antibody level).

FIG. 21: Study flowchart. PBDD: post blood draw delay; PAP: post-acute phase.

FIG. 22: Performance characteristics of the NETs biomarkers for COVID-19 (example 17). ROC curves for NE, MPO, cir-nDNA, cir-mtDNA concentrations and MNR between healthy individuals and COVID-19 patients (NS, S, and PAP). ROC curves of these markers in combining both NS and S COVID-19 patient cohorts vs HI (AUC of 0.97, 0.99, 0.98, and 1.0 for NE, MPO, cir-nDNA, and MNR, respectively). AUC determined in the PAP cohort when using NE, MPO, cir-nDNA, cir-mtDNA concentrations and MNR, are 0.64, 0.82, 0.93, 0.70, and 0.84, respectively.ROC: receiver operating characteristics; AUC: area under curve; NE: neutrophil elastase; MPO: myeloperoxidase; cirDNA: circulating cell-free DNA; cir-mtDNA: circulating cell-free DNA of mitochondrial origin; MNR: ratio of mitochondrial to nuclear circulating DNA concentration.

FIG. 23: Illustration of the higher diagnostic capacity when combining NETs and cirDNA markers thresholds (Exemple 18)

ROC and AUC determined in the severe cohort from the NE values alone with concentration higher than 21 ng/mL, from the cir-nDNA values alone with concentration higher than 9 ng/ml, and from both NE and cir-nDNA values higher than 21 and 9 ng/mL, respectively. Combining NE and cir-nDNA thresholds provides an AUC of 0.998 as compared to 0.953 and 0.940 in NE alone and cir-nDNA alone, respectively. NE, NE threshold taken independently; MPO, cir-nDNA threshold taken independently; and NE+cir-nDNA, NE and cir-DNA thresholds being combined.

TABLE 1
Values of the qPCR and ELISA analysis from COVID-19 patients and healthy

| | | | | Plasma after 16000 g | | | Plasma after 1200 g | | |
|---|---|---|---|---|---|---|---|---|---|
| | | | | cir- | cir- | | cir- | cir- | |
| | | NE, | MPO, | nDNA, | mtDNA, | | nDNA, | mtDNA, | |
| Patients | Sample | ng/ml | ng/ml | ng/ml | ng/ml | MNR | ng/ml | ng/ml | MNR |
| COVID 19 | R3 | 108.2 | 184.9 | 529 | 1.106 | 2.00E−04 | 1076.5 | 1.6 | 1.47E−0 |
| in ICU | R4 | 126.7 | 181.2 | 458 | 0.473 | 1.03E−03 | 949.6 | 5.5 | 5.76E−0 |
| (N = 18) | R6 | 86.7 | 98.3 | 334 | 0.154 | 4.61E−04 | 448.7 | 1.4 | 3.23E−0 |
| | R7 | 78.8 | 172.2 | 84 | 0.019 | 2.27E−04 | 98.2 | 0.7 | 6.72E−0 |
| | R8 | 71.7 | 83.4 | 312 | 0.178 | 5.71E−04 | 254.4 | 3.1 | 1.22E−0 |
| | R9 | 31.2 | 112.9 | 196 | 0.059 | 2.98E−04 | 329.8 | 0.8 | 2.28E−0 |
| | R11 | 44.7 | 55.2 | 76 | 0.013 | 1.67E−04 | 77.6 | 0.8 | 9.68E−0 |
| | R12 | 46.0 | 56.7 | 76 | 0.008 | 1.00E−04 | 94.1 | 1.5 | 1.58E−0 |
| | R15 | 28.1 | 113.9 | 305 | 0.037 | 1.23E−04 | 520.9 | 3.0 | 5.76E−0 |
| | R16 | 194.3 | 111.1 | 114 | 0.022 | 1.96E−04 | 196.6 | 4.2 | 2.13E−0 |
| | R19 | 202.0 | 194.0 | 239 | 0.036 | 1.49E−04 | 325.0 | 1.8 | 5.51E−0 |
| | R20 | 168.5 | 172.1 | 885 | 0.112 | 1.27E−04 | 1326.4 | 2.5 | 1.91E−0 |
| | R21 | 70.9 | 130.6 | 55 | 0.011 | 1.91E−04 | 125.5 | 6.2 | 4.92E−0 |
| | R32 | 41.7 | 160.6 | 165 | 0.046 | 2.82E−04 | 312.8 | 2.0 | 6.26E−0 |
| | P1 | 62.4 | 48.4 | 225 | 0.019 | 8.43E−04 | | | |
| | P2 | 55.2 | 102.5 | 151 | 0.053 | 3.52E−04 | | | |
| | P3 | 47.3 | 102.9 | 75 | 0.114 | 1.52E−04 | | | |
| | P4 | 62.8 | 91.7 | 145 | 0.048 | 3.31E−04 | | | |
| | MEAN | 84.8 | 120.7 | 245.8 | 0.084 | 3.56E−04 | 438.3 | 2.5 | 1.05E−0 |
| | MEDIAN | 66.8 | 112.0 | 180.4 | 0.047 | 2.13E−04 | 318.9 | 1.9 | 6.01E−0 |
| | SD | 54.1 | 46.9 | 209.3 | 0.110 | 3.67E−04 | 398.0 | 1.7 | 1.25E−0 |
| COVID 19 | M9 | 60.1 | 106.9 | 110 | 0.016 | 1.46E−04 | 133.7 | 1.5 | 1.11E−0 |
| hospitalized | M12 | 62.3 | 68.3 | 234 | 0.023 | 9.80E−05 | 283.3 | 2.9 | 1.04E−0 |
| (N = 14) | M15 | 32.7 | 50.1 | 57 | 0.007 | 1.32E−04 | 117.7 | 2.1 | 1.78E−0 |
| | M18 | 44.6 | 74.3 | 264 | 0.063 | 2.38E−04 | 539.7 | 5.1 | 9.41E−0 |
| | M21 | 14.5 | 26.1 | 17 | 0.019 | 1.08E−03 | 38.6 | 1.1 | 2.81E−0 |
| | M35 | 14.6 | 30.2 | 5 | 0.003 | 7.02E−04 | 15.0 | 2.3 | 1.56E−0 |
| | M38 | 33.9 | 74.8 | 118 | 0.011 | 9.43E−05 | 120.6 | 2.9 | 2.38E−0 |
| | M39 | 34.9 | 42.5 | 7 | 0.001 | 8.50E−05 | 9.1 | 0.1 | 6.01E−0 |
| | M40 | 45.9 | 104.4 | 93 | 0.019 | 2.08E−04 | 101.1 | 1.0 | 9.52E−0 |
| | M41 | 28.1 | 75.2 | 128 | 0.012 | 9.55E−05 | 137.8 | 7.8 | 5.66E−0 |
| | M44 | 13.3 | 26.0 | 9 | 0.009 | 9.44E−05 | 6.6 | 0.6 | 9.59E−0 |
| | M45 | 56.3 | 117.2 | 188 | 0.044 | 2.32E−04 | 279.4 | 0.6 | 2.27E−0 |
| | M50 | 18.1 | 68.2 | 71 | 0.017 | 2.43E−04 | 51.9 | 0.7 | 1.42E−0 |
| | M52 | 22.5 | 45.9 | 11 | 0.005 | 4.69E−04 | 14.2 | 1.7 | 1.21E−0 |
| | MEAN | 34.4 | 65.0 | 93.8 | 0.018 | 3.41E−04 | 132.1 | 2.2 | 4.02E−0 |
| | MEDIAN | 33.3 | 68.3 | 82.1 | 0.014 | 2.20E−04 | 109.4 | 1.6 | 1.60E−0 |
| | SD | 17.2 | 30.0 | 86.2 | 0.017 | 3.33E−04 | 148.3 | 2.1 | 4.90E−0 |
| Healthy | MEAN | 14.5 | 13.6 | 6.0 | 0.405 | 9.63E−02 | 5.28 | 0.73 | 1.44E−0 |
| | MEDIAN | 12.9 | 11.8 | 5.8 | 0.277 | 5.77E−02 | 5.05 | 0.82 | 4.42E−0 |
| | SD | 8.3 | 9.5 | 2.3 | 0.373 | 1.12E−01 | 1.15 | 0.28 | 6.01E−0 |

TABLE 2
Summary of NE, MPO, cir-nDNA, cir-mtDNA, MNR and exMT

| | NE, ng/ml | | | MPO, ng/ml | | | cir-nDNA, ng/ml | | |
|---|---|---|---|---|---|---|---|---|---|
| | | | COVID- | | | COVID- | | | COVID- |
| | | | 19 | | | 19 | | | 19 |
| | | COVID-19 | in | | COVID-19 | in | | COVID-19 | in |
| | HEALTHY | hospitalized | ICU | HEALTHY | hospitalized | ICU | HEALTHY | hospitalized | ICU |
| Mean | 14.50 | 34.4 | 84.8 | 13.63 | 65.0 | 120.7 | 5.97 | 93.71 | 245.8 |
| Median | 12.87 | 33.3 | 66.8 | 11.79 | 68.3 | 112.0 | 5.78 | 82 | 180.4 |
| SD | 8.31 | 17.2 | 54.1 | 9.54 | 30.0 | 46.9 | 2.34 | 86.27 | 209.3 |
| | cir-mtDNA, ng/ml | | | MNR | | | exMT | | |
| | | | COVID- | | | COVID- | | | COVID- |
| | | | 19 | | | 19 | | | 19 |
| | | COVID-19 | in | | COVID-19 | in | | COVID-19 | in |
| | HEALTHY | hospitalized | ICU | HEALTHY | hospitalized | ICU | HEALTHY | hospitalized | ICU |
| Mean | 0.405 | 0.018 | 0.084 | 9.63E−02 | 3.41E−04 | 3.56E−04 | 1.2 | 2.2 | 2.4 |
| Median | 0.277 | 0.014 | 0.047 | 5.77E−02 | 2.20E−04 | 2.13E−04 | 1.4 | 1.6 | 1.8 |
| SD | 0.373 | 0.017 | 0.110 | 1.12E−01 | 3.33E−04 | 3.67E−04 | 0.5 | 2.1 | 1.7 |

TABLE 3
Statistical analysis of the data

| HEALTHY | | | | | | | | | |
|---|---|---|---|---|---|---|---|---|---|
| vs | | | | Mean | | | | | |
| COVID- | | | Mean | COVID- | | SE of | | | |
| 19 ALL | Significant? | P value | HEALTHY | 19 ALL | Difference | difference | t ratio | df | Ad |
| NE | Yes | <0.000001 | 14.5 | 62.78 | −48.28 | 4.777 | 10.11 | 143 | <0 |
| MPO | Yes | <0.000001 | 13.63 | 96.33 | −82.7 | 4.843 | 17.08 | 143 | <0 |
| cirDNA | Yes | <0.000001 | 5.972 | 179.2 | −173.3 | 16.95 | 10.22 | 143 | <0 |
| cir-mtDNA | Yes | 0.000003 | 0.405 | 0.060 | 0.3444 | 0.06783 | 5.078 | 65 | 0. |
| MNR | Yes | 0.000009 | 95345 | 375.1 | 94970 | 19680 | 4.826 | 65 | 0. |
| exMT | No | 0.211348 | 1.200 | 2.280 | −1.08 | 0.846 | 1.276 | 31 | 0. |
| HEALTHY | | | | Mean | | | | | |
| vs | | | | COVID- | | | | | |
| COVID-19 | | | Mean | 19 | | SE of | | | |
| Hospitalized | Significant? | P value | HEALTHY | Hospitalized | Difference | difference | t ratio | df | Ad |
| NE | Yes | <0.000001 | 14.5 | 34.41 | −19.91 | 2.727 | 7.303 | 125 | <0 |
| MPO | Yes | <0.000001 | 13.63 | 65.01 | −51.37 | 3.748 | 13.71 | 125 | <0 |
| cirDNA | Yes | <0.000001 | 5.972 | 93.71 | −87.74 | 7.908 | 11.1 | 125 | <0 |
| cir-mtDNA | Yes | 0.000349 | 0.405 | 0.018 | 0.3869 | 0.1003 | 3.856 | 47 | 0. |
| MNR | Yes | 0.002643 | 95345 | 337 | 95008 | 29922 | 3.175 | 47 | 0. |
| exMT | No | 0.328796 | 1.200 | 2.157 | −0.9569 | 0.9518 | 1.005 | 17 | 0. |
| HEALTHY | | | | Mean | | | | | |
| vs | | | | COVID- | | | | | |
| COVID-19 | | | Mean | 19 | | SE of | | | |
| in ICU | Significant? | P value | HEALTHY | in ICU | Difference | difference | t ratio | df | Ad |
| NE | Yes | <0.000001 | 14.5 | 84.84 | −70.34 | 5.358 | 33.13 | 129 | <0 |
| MPO | Yes | <0.000001 | 13.63 | 120.7 | −107.1 | 4.871 | 21.98 | 129 | <0 |
| cirDNA | Yes | <0.000001 | 5.972 | 245.8 | −239.8 | 19.29 | 12.43 | 129 | <0 |
| cir-mtDNA | Yes | 0.001147 | 0.405 | 0.093 | 0.3114 | 0.09037 | 3.446 | 51 | 0. |
| MNR | Yes | 0.000712 | 95345 | 404.7 | 94940 | 26347 | 3.603 | 51 | 0. |
| exMT | No | 0.141779 | 1.200 | 2.402 | −1.202 | 0.7804 | 1.541 | 17 | 0. |
| COVID-19: | | | Mean | Mean | | | | | |
| Hospitalized | | | COVID- | COVID- | | | | | |
| vs | | | 19 | 19 | | SE of | | | |
| in ICU | Significant? | P value | in ICU | Hospitalized | Difference | difference | t ratio | df | Ad |
| NE | Yes | 0.002205 | 84.84 | 34.41 | 50.43 | 15.06 | 3.348 | 30 | 0. |
| MPO | Yes | 0.00055 | 120.7 | 65.01 | 55.69 | 14.4 | 3.867 | 30 | 0 |
| cirDNA | Yes | 0.016207 | 245.8 | 93.71 | 152.1 | 59.69 | 2.548 | 30 | 0. |
| cir-mtDNA | No | 0.020616 | 0.093 | 0.018 | 0.07551 | 0.0309 | 2.444 | 30 | 0. |
| MNR | No | 0.62223 | 404.7 | 337 | 67.68 | 135.9 | 0.4978 | 30 | 0 |
| exMT | No | 0.733373 | 2.157 | 2.402 | −0.2455 | 0.713 | 0.3443 | 26 | 0 |

TABLE 4
Comparison of COVID-19 hospitalized and
ICU patients vs healthy individuals

| | Median | | | | |
|---|---|---|---|---|---|
| | Elastase | MPO | Cir-nDNA | Cir-mtDNA | |
| | ng/mL | ng/mL | ng/mL | ng/mL | MNR |
| Healthy | 12.90 | 11.80 | 5.80 | 0.28 | 0.05 |
| COVID-19, | 33.40 | 68.40 | 82.10 | 0.01 | 0.00 |
| hospitalized | | | | | |
| COVID-19, | 75.30 | 122.30 | 217.80 | 0.04 | 0.00 |
| ICU | | | | | |

| | Fold increase vs healthy subjects | | | | |
|---|---|---|---|---|---|
| COVID-19, | 3-fold | 6-fold | 16-fold | 20-fold | 150-fold |
| hospitalized | | | | less | less |
| COVID-19, ICU | 6-fold | 12-fold | 43-fold | 7-fold less | 150-fold |
| | | | | | less |

TABLE 5
Selected ratio involving cir-nDNA discriminating
ICU vs hospitalized patients

| | | Values for cir-nDNA | | |
|---|---|---|---|---|
| | | (Plasma after 16000 g) | | |
| | | NE/cir- | MPO/cir- | NE × MPO/ |
| Patients | Sample | nDNA | nDNA | cir-nDNA |
| COVID 19 in ICU | R3 | 0.20 | 0.35 | 0.07 |
| (N = 18) | R4 | 0.28 | 0.40 | 0.11 |
| | R6 | 0.26 | 0.29 | 0.08 |
| | R7 | 0.94 | 2.05 | 1.93 |
| | R8 | 0.23 | 0.27 | 0.06 |
| | R9 | 0.16 | 0.57 | 0.09 |
| | R11 | 0.59 | 0.73 | 0.43 |
| | R12 | 0.60 | 0.74 | 0.45 |
| | R15 | 0.09 | 0.37 | 0.03 |
| | R16 | 1.70 | 0.97 | 1.66 |
| | R19 | 0.84 | 0.81 | 0.68 |
| | R20 | 0.19 | 0.19 | 0.04 |
| | R21 | 1.29 | 2.37 | 3.06 |
| | R32 | 0.25 | 0.98 | 0.25 |
| | P1 | 0.28 | 0.21 | 0.06 |
| | P2 | 0.37 | 0.68 | 0.25 |
| | P3 | 0.63 | 1.37 | 0.86 |
| | P4 | 0.43 | 0.63 | 0.27 |
| | MEAN | 0.52 | 0.78 | 0.58 |
| | MEDIAN | 0.32 | 0.66 | 0.25 |
| | SD | 0.43 | 0.61 | 0.83 |
| COVID 19 hospitalized | M9 | 0.54 | 0.97 | 0.53 |
| (N = 14) | M12 | 0.27 | 0.29 | 0.08 |
| | M15 | 0.58 | 0.89 | 0.51 |
| | M18 | 0.17 | 0.28 | 0.05 |
| | M21 | 0.83 | 1.50 | 1.25 |
| | M35 | 2.93 | 6.07 | 17.79 |
| | M38 | 0.29 | 0.63 | 0.18 |
| | M39 | 4.77 | 5.80 | 27.67 |
| | M40 | 0.49 | 1.12 | 0.56 |
| | M41 | 0.22 | 0.59 | 0.13 |
| | M44 | 1.43 | 2.79 | 3.99 |
| | M45 | 0.30 | 0.62 | 0.19 |
| | M50 | 0.25 | 0.96 | 0.24 |
| | M52 | 2.03 | 4.14 | 8.41 |
| | MEAN | 1.08 | 1.90 | 4.40 |
| | MEDIAN | 0.52 | 0.96 | 0.52 |
| | SD | 1.33 | 2.00 | 8.32 |

| | | | |
|---|---|---|---|
| P value: Hosp vs ICU | 0.104 | 0.031 | 0.061 |
| median healthy | 2.22 | 2.0 | 4.44 |
| diagnostic power | * | * | * |

TABLE 6
Selected ratio involving cir-mtDNA discriminating
ICU vs hospitalized patients

| | | Values for cir-mtDNA | | |
|---|---|---|---|---|
| | | (Plasma after 16000 g) | | |
| | | NE/cir- | MPO/cir- | NE × MPO/ |
| Patients | Sample | mtDNA | mtDNA | cir-mtDNA |
| COVID 19 in ICU | R3 | 1.02E+03 | 1.75E+03 | 1.79E+06 |
| (N = 18) | R4 | 2.68E+02 | 3.84E+02 | 1.03E+05 |
| | R6 | 5.63E+02 | 6.38E+02 | 3.60E+05 |
| | R7 | 4.14E+03 | 9.05E+03 | 3.75E+07 |
| | R8 | 4.03E+02 | 4.68E+02 | 1.89E+05 |
| | R9 | 5.33E+02 | 1.93E+03 | 1.03E+06 |
| | R11 | 3.52E+03 | 4.35E+03 | 1.53E+07 |
| | R12 | 6.02E+03 | 7.42E+03 | 4.46E+07 |
| | R15 | 7.51E+02 | 3.04E+03 | 2.29E+06 |
| | R16 | 8.71E+03 | 4.98E+03 | 4.34E+07 |
| | R19 | 5.66E+03 | 5.44E+03 | 3.08E+07 |
| | R20 | 1.50E+03 | 1.54E+03 | 2.31E+06 |
| | R21 | 6.75E+03 | 1.24E+04 | 8.39E+07 |
| | R32 | 9.00E+02 | 3.47E+03 | 3.12E+06 |
| | P1 | 3.28E+03 | 2.55E+03 | 8.37E+06 |
| | P2 | 1.04E+03 | 1.93E+03 | 2.01E+06 |
| | P3 | 4.15E+02 | 9.02E+02 | 3.74E+05 |
| | P4 | 1.31E+03 | 1.91E+03 | 2.50E+06 |
| | MEAN | 2.60E+03 | 3.57E+03 | 1.56E+07 |
| | MEDIAN | 1.17E+03 | 2.24E+03 | 2.40E+06 |
| | SD | 2.62E+03 | 3.27E+03 | 2.33E+07 |
| COVID 19 | M9 | 3.73E+03 | 6.64E+03 | 2.48E+07 |
| hospitalized | M12 | 2.72E+03 | 2.98E+03 | 8.11E+06 |
| (N = 14) | M15 | 4.37E+03 | 6.69E+03 | 2.93E+07 |
| | M18 | 7.10E+02 | 1.18E+03 | 8.41E+05 |
| | M21 | 7.68E+02 | 1.39E+03 | 1.06E+06 |
| | M35 | 4.17E+03 | 8.66E+03 | 3.61E+07 |
| | M38 | 3.05E+03 | 6.71E+03 | 2.04E+07 |
| | M39 | 5.61E+04 | 6.83E+04 | 3.83E+09 |
| | M40 | 2.37E+03 | 5.40E+03 | 1.28E+07 |
| | M41 | 2.31E+03 | 6.17E+03 | 1.42E+07 |
| | M44 | 1.51E+03 | 2.96E+03 | 4.47E+06 |
| | M45 | 1.29E+03 | 2.68E+03 | 3.46E+06 |
| | M50 | 1.05E+03 | 3.93E+03 | 4.11E+06 |
| | M52 | 4.34E+03 | 8.83E+03 | 3.83E+07 |
| | MEAN | 6.32E+03 | 9.46E+03 | 2.88E+08 |
| | MEDIAN | 2.55E+03 | 5.79E+03 | 1.35E+07 |
| | SD | 1.44E+04 | 1.71E+04 | 1.02E+09 |

| | | | |
|---|---|---|---|
| P value: Hosp vs ICU | 0.289 | 0.161 | 0.264 |
| median healthy | 46.6 | 42.6 | 9.20E+04 |
| diagnostic power | * | ** | *** |

TABLE 7
NE, MPO, cir-nDNA and DII values as
determined in 10 mCRC patients.

| | MPO | Ela2 | Cir-nDNA | DII |
|---|---|---|---|---|
| PATIENT | (ng/mL) | (ng/mL) | (ng/mL) | Integrity |
| ID | Plasma | Plasma | plasma | Index |
| #S07 025 | 73.3 | 69.6 | 111.0 | 0.021 |
| #S13 002 | 103.5 | 81.5 | 50.2 | 0.017 |
| #S29 004 | 51.6 | 22.4 | 19.2 | 0.003 |
| #S20 002 | 29.3 | 3.9 | 13.9 | 0.000 |
| #S02 015 P2 | 50.9 | 28.7 | 59.0 | 0.000 |
| #S09 005 | 84.3 | 57.8 | 45.3 | 0.000 |
| #S13 003 | 56.7 | 71.1 | 22.3 | 0.058 |
| #S07 022 | 174.3 | 103.6 | 62.2 | 0.062 |
| #S02 014 | 126.5 | 74.7 | 67.9 | 0.010 |
| #S07 016 | 31.7 | 54.5 | 8.3 | 0.007 |
| Moyenne | 78.2 | 56.8 | 45.9 | 0.018 |
| SD | 43.3 | 28.7 | 29.8 | 0.022 |

SD, Standard deviation.

TABLE 8
Statistical analysis of the comparison of the values obtained from the mCRC
patients and healthy individuals when analysing NE, MPO, and cir-nDNA

| | | | | Mean | Mean | | SE of | t | | Adjusted |
|---|---|---|---|---|---|---|---|---|---|---|
| | | Significant? | P value | mCRC | EFS | Difference | difference | ratio | df | P Value |
| MCRC | NE | Yes | <0.0001 | 56.78 | 14.5 | 42.28 | 3.794 | 11.14 | 121 | <0.0001 |
| vs EFS | MPO | Yes | <0.0001 | 78.21 | 13.63 | 64.57 | 5.102 | 12.66 | 121 | <0.0001 |
| | cirDNA | Yes | <0.0001 | 45.92 | 5.972 | 39.95 | 2.921 | 13.67 | 121 | <0.0001 |

TABLE 9A
Non-sévère (n = 26)

| | NE | MPO | cir-nDNA | cir-mtDNA | |
|---|---|---|---|---|---|
| Sample | [ng/ml] | [ng/ml] | [ng/ml] | [ng/ml] | MNR |
| M09 | 60.1 | 106.9 | 110 | 0.016 | 1.45E−04 |
| M11 | 25.9 | 85.3 | 62 | 0.004 | 6.45E−05 |
| M14 | 40 | 79.4 | 28 | 0.004 | 1.43E−04 |
| M15 | 32.7 | 50.1 | 57 | 0.007 | 1.23E−04 |
| M17 | 72.4 | 127.9 | 119 | 0.017 | 1.43E−04 |
| M18 | 44.6 | 74.3 | 264 | 0.063 | 2.39E−04 |
| M25 | 59.2 | 64 | 41 | 0.005 | 1.22E−04 |
| M30 | 46 | 43.1 | 67 | 0.005 | 7.46E−05 |
| M35 | 14.6 | 30.2 | 5 | 0.003 | 6.00E−04 |
| M39 | 34.9 | 42.5 | 7 | 0.001 | 1.43E−04 |
| M40 | 45.9 | 104.4 | 93 | 0.019 | 2.04E−04 |
| M41 | 28.1 | 75.2 | 128 | 0.012 | 9.38E−05 |
| M45 | 56.3 | 117.2 | 188 | 0.044 | 2.34E−04 |
| M50 | 18.1 | 68.2 | 71 | 0.017 | 2.39E−04 |
| M51 | 36.2 | 107.3 | 137 | 0.015 | 1.09E−04 |
| M52 | 22.5 | 45.9 | 11 | 0.005 | 4.55E−04 |
| M53 | 62.6 | 57.5 | 73 | 0.005 | 6.85E−05 |
| M54 | 29.8 | 39.9 | 166 | 0.011 | 6.63E−05 |
| M55 | 77.1 | 61.7 | 14 | 0.016 | 1.14E−03 |
| M60 | 45.6 | 74.9 | 23 | 0.005 | 2.17E−04 |
| M61 | 22.5 | 39.5 | 6 | 0.033 | 5.50E−03 |
| M65 | 31.7 | 50.5 | 206 | 0.032 | 1.55E−04 |
| M68 | 46.7 | 64.5 | 18 | 0.022 | 1.22E−03 |
| M69 | 56.3 | 43.9 | 190 | 0.015 | 7.89E−05 |
| M76 | 45.1 | 60.9 | 70 | 0.072 | 1.03E−03 |
| M81 | 40.4 | 67.3 | 27 | 0.004 | 1.48E−04 |

TABLE 9B
Sévère (n = 44)

| | NE | MPO | cir-nDNA | cir-mtDNA | |
|---|---|---|---|---|---|
| Sample | [ng/ml] | [ng/ml] | [ng/ml] | [ng/ml] | MNR |
| M12 | 62.3 | 68.3 | 234 | 0.023 | 9.83E−05 |
| M21 | 14.5 | 26.1 | 17 | 0.019 | 1.12E−03 |
| M38 | 33.9 | 74.8 | 118 | 0.011 | 9.32E−05 |
| M44 | 13.3 | 26 | 9 | 0.009 | 1.00E−03 |
| M49 | | | 32 | 0.019 | 5.94E−04 |
| M56 | 51.7 | 76.9 | 34 | 0.018 | 5.29E−04 |
| M74 | 63.6 | 114.4 | 221 | 0.02 | 9.05E−05 |
| M82 | 81.1 | 125.2 | 133 | 0.017 | 1.28E−04 |
| M83 | 70.1 | 79 | 132 | 0.009 | 6.82E−05 |
| R01 | 76.5 | 136 | 26 | 0.024 | 9.23E−04 |
| R03 | 108.2 | 184.9 | 529 | 0.106 | 2.00E−04 |
| R04 | 126.7 | 181.2 | 458 | 0.473 | 1.03E−03 |
| R06 | 86.7 | 98.3 | 334 | 0.154 | 4.61E−04 |
| R08 | 71.7 | 83.4 | 312 | 0.178 | 5.71E−04 |
| R09 | 31.2 | 112.9 | 196 | 0.059 | 3.01E−04 |
| R11 | 44.7 | 55.2 | 76 | 0.013 | 1.71E−04 |
| R12 | 46 | 56.7 | 76 | 0.008 | 1.05E−04 |
| R13 | 159.9 | 205.1 | 81 | 0.038 | 4.69E−04 |
| R15 | 28.1 | 113.9 | 305 | 0.037 | 1.21E−04 |
| R16 | 194.3 | 111.1 | 114 | 0.022 | 1.93E−04 |
| R19 | 202 | 194 | 239 | 0.036 | 1.51E−04 |
| R20 | 168.5 | 172.1 | 885 | 0.112 | 1.27E−04 |
| R21 | 70.9 | 130.6 | 55 | 0.011 | 2.00E−04 |
| R22 | 51.8 | 105.3 | 38 | 0.056 | 1.47E−03 |
| R23 | 124.4 | 140.8 | 68 | 0.013 | 1.91E−04 |
| R24 | 122.1 | 116.4 | 91 | 0.033 | 3.63E−04 |
| R25 | 172.7 | 143.7 | 226 | 0.072 | 3.19E−04 |
| R26 | 172.3 | 199.8 | 79 | 0.086 | 1.09E−03 |
| R29 | 63.3 | 125.2 | 155 | 0.017 | 1.10E−04 |
| R32 | 41.7 | 160.6 | 165 | 0.046 | 2.79E−04 |
| R33 | 97.8 | 115.4 | 366 | 0.086 | 2.35E−04 |
| R35 | 85.3 | 94.3 | 199 | 0.027 | 1.36E−04 |
| R36 | 117.8 | 199 | 48 | 0.079 | 1.65E−03 |
| R37 | 74.6 | 93.9 | 207 | 0.024 | 1.16E−04 |
| R38 | 115.2 | 85.1 | 328 | 0.012 | 3.66E−05 |
| R39 | 128.5 | 75.3 | 21 | 0.003 | 1.43E−04 |
| R42 | 87.4 | 63.6 | 137 | 0.04 | 2.92E−04 |
| R44 | 48.3 | 88.9 | 22 | 0.007 | 3.18E−04 |
| R46 | 104.5 | 199.8 | 167 | 0.059 | 3.53E−04 |
| R49 | 43.4 | 77 | 72 | 0.021 | 2.92E−04 |
| R51 | 64.8 | 115.5 | 476 | 0.037 | 7.77E−05 |
| R53 | 36.4 | 109.6 | 123 | 0.032 | 2.60E−04 |
| R55 | 132.7 | 193.9 | 800 | 0.276 | 3.45E−04 |
| R56 | 48 | 81 | 349 | 0.056 | 1.60E−04 |

TABLE 9C
Healthy (n = 119)

| | NE | MPO | cir-nDNA | cir-mtDNA | |
|---|---|---|---|---|---|
| Sample | [ng/ml] | [ng/ml] | [ng/ml] | [ng/ml] | MNR |
| EFS-001 | 22.74 | 25.28 | 10.12 | 0.17 | 1.68E−02 |
| EFS-0203 | 22.21 | 15.2 | 5.69 | 0.116 | 2.04E−02 |
| EFS-022 | 14.89 | 10.32 | 9.182 | 0.0237 | 2.58E−03 |
| EFS-0238 | 15.51 | 15.01 | 8.124 | 0.0587 | 7.23E−03 |
| EFS-0246 | 9.26 | 8.95 | 8.253 | 0.0554 | 6.71E−03 |
| EFS-0254 | 25.51 | 23.38 | 7.805 | 0.0238 | 3.05E−03 |
| EFS-0326 | 19.08 | 14.79 | 6.136 | 0.107 | 1.74E−02 |
| EFS-0481 | 11.61 | 7.4 | 9.772 | 0.0958 | 9.80E−03 |
| EFS-049 | 12.06 | 12.81 | 6.853 | 0.0877 | 1.28E−02 |
| EFS-0675 | 24.29 | 15.2 | 2.556 | 0.0238 | 9.31E−03 |
| EFS-0908 | 18.08 | 18.17 | 9.021 | 0.129 | 1.43E−02 |
| EFS-0975 | 16.32 | 6.53 | 4.345 | 0.107 | 2.46E−02 |
| EFS-1040 | 14.39 | 16.45 | 3.206 | 0.0697 | 2.17E−02 |
| EFS-1059 | 13.08 | 10.1 | 2.801 | 0.122 | 4.36E−02 |
| EFS-1067 | 9.02 | 15.25 | 3.937 | 0.202 | 5.13E−02 |
| EFS-1150 | 10.84 | 6.43 | 7.481 | 0.032 | 4.28E−03 |
| EFS-1653 | 12.32 | 15.18 | 4.51 | 0.0533 | 1.18E−02 |
| EFS-167- | 10.11 | 11.74 | 6.88 | 1.22 | 1.77E−01 |
| EFS-1688 | 10.66 | 14.56 | 4.879 | 0.0847 | 1.74E−02 |
| EFS-1717 | 11.64 | 16.28 | 2.425 | 0.106 | 4.37E−02 |
| EFS-1783 | 8 | 5 | 7.214 | 0.102 | 1.41E−02 |
| EFS-1791 | 10.9 | 9.11 | 4.948 | 0.119 | 2.41E−02 |
| EFS-1804 | 11.96 | 7.12 | 4.575 | 0.22 | 4.81E−02 |
| EFS-1812 | 9.52 | 7.32 | 8.749 | 0.166 | 1.90E−02 |
| EFS-2015 | 15.53 | 13.95 | 2.471 | 0.0936 | 3.79E−02 |
| EFS-2058 | 12.87 | 12.93 | 5.418 | 0.0999 | 1.84E−02 |
| EFS-2066 | 12.88 | 20.54 | 8.177 | 0.38 | 4.65E−02 |
| EFS-2254 | 9.43 | 12.98 | 5.515 | 0.446 | 8.09E−02 |
| EFS-2285 | 7.35 | 8.58 | 2.59 | 0.218 | 8.42E−02 |
| EFS-2293 | 7.99 | 11.41 | 3.01 | 0.383 | 1.27E−01 |
| EFS-2318 | 11.49 | 14 | 6.268 | 0.578 | 9.22E−02 |
| EFS-2322 | 8.89 | 14.4 | 4.931 | 0.246 | 4.99E−02 |
| EFS-2657 | 15.97 | 8.35 | 4.092 | 0.0162 | 3.96E−03 |
| EFS-2673 | 18.02 | 6.53 | 5.377 | 0.0519 | 9.65E−03 |
| EFS-269- | 12.81 | 11.25 | 3.123 | 0.0697 | 2.23E−02 |
| EFS-2780 | 7.82 | 15.48 | 5.02 | 0.501 | 9.98E−02 |
| EFS-2801 | 7.11 | 11.76 | 3.112 | 0.309 | 9.93E−02 |
| EFS-2844 | 11.42 | 12.18 | 2.119 | 0.0578 | 2.73E−02 |
| EFS-2852 | 10.76 | 9.86 | 2.355 | 0.0446 | 1.89E−02 |
| EFS-2875 | 16.54 | 22.03 | 3.541 | 0.874 | 2.47E−01 |
| EFS-2891 | 8.77 | 4.91 | 5.212 | 0.958 | 1.84E−01 |
| EFS-3295 | 43.43 | 24.98 | 9.617 | 0.467 | 4.86E−02 |
| EFS-3316 | 11.17 | 10.8 | 4.138 | 0.0957 | 2.31E−02 |
| EFS-3359 | 10.11 | 8.75 | 6.046 | 0.106 | 1.75E−02 |
| EFS-3367 | 19.77 | 13.14 | 7.832 | 0.0298 | 3.80E−03 |
| EFS-3375 | 14.08 | 4.22 | 9.699 | 0.0477 | 4.92E−03 |
| EFS-3391 | 13.34 | 8.01 | 7.753 | 0.048 | 6.19E−03 |
| EFS-3463 | 17.24 | 6.32 | 5.163 | 0.0157 | 3.04E−03 |
| EFS-3471 | 12.8 | 12.51 | 4.205 | 0.014 | 3.33E−03 |
| EFS-348- | 10.22 | 8.54 | 9.42 | 0.0446 | 4.73E−03 |
| EFS-372- | 3.27 | 2.221 | 4.306 | 0.154 | 3.58E−02 |
| EFS-3796 | 7.65 | 13.09 | 1.297 | 0.512 | 3.95E−01 |
| EFS-3975 | 9.32 | 3.871 | 5.577 | 0.496 | 8.89E−02 |
| EFS-3983 | 14.95 | 8.45 | 4.061 | 0.032 | 7.88E−03 |
| EFS-3991 | 17.47 | 7.53 | 5.152 | 0.0361 | 7.01E−03 |
| EFS-4003 | 16.72 | 14.84 | 2.612 | 0.0181 | 6.93E−03 |
| EFS-4167 | 4.41 | 5.57 | 3.766 | 0.0859 | 2.28E−02 |
| EFS-4175 | 3.67 | 16.17 | 5.069 | 0.19 | 3.75E−02 |
| EFS-4204 | 4.15 | 11.42 | 7.477 | 0.186 | 2.49E−02 |
| EFS-4212 | 15.65 | 8.34 | 6.215 | 0.0903 | 1.45E−02 |
| EFS-4220 | 18.75 | 13.31 | 8.617 | 0.105 | 1.22E−02 |
| EFS-4239 | 10.95 | 8.78 | 5.775 | 0.279 | 4.83E−02 |
| EFS-4268 | 8.96 | 10.43 | 2.664 | 1.51 | 5.67E−01 |
| EFS-4276 | 10.11 | 13.29 | 1.382 | 0.758 | 5.48E−01 |
| EFS-4292 | 12.59 | 12.54 | 4.654 | 0.496 | 1.07E−01 |
| EFS-4321 | 13.31 | 12.23 | 6.63 | 1.04 | 1.57E−01 |
| EFS-4326 | 47.17 | 73.02 | 2.829 | 0.0708 | 2.50E−02 |
| EFS-4540 | 9.92 | 12.03 | 3.832 | 0.277 | 7.23E−02 |
| EFS-4650 | 20.8 | 12.44 | 4.908 | 0.293 | 5.97E−02 |
| EFS-4687 | 8.7 | 14.86 | 7.778 | 0.0861 | 1.11E−02 |
| EFS-4724 | 17.58 | 11.7 | 4.619 | 0.0478 | 1.03E−02 |
| EFS-4759 | 21.06 | 19.2 | 6.012 | 0.0354 | 5.89E−03 |
| EFS-4767 | 16.45 | 12.19 | 7.852 | 0.0401 | 5.11E−03 |
| EFS-4941 | 12.23 | 8.06 | 8.905 | 0.616 | 6.92E−02 |
| EFS-495- | 14.55 | 20.4 | 7.542 | 0.759 | 1.01E−01 |
| EFS-4977 | 7.53 | 14.33 | 12.258 | 0.226 | 1.84E−02 |
| EFS-4984 | 10.93 | 8.02 | 7.336 | 0.0154 | 2.10E−03 |
| EFS-4985 | 13.09 | 20.4 | 5.676 | 0.244 | 4.30E−02 |
| EFS-4993 | 8.16 | 14.07 | 5.26 | 0.395 | 7.51E−02 |
| EFS-5012 | 9.95 | 8.14 | 6.597 | 0.626 | 9.49E−02 |
| EFS-5190 | 63.9 | 73.52 | 2.925 | 0.0326 | 1.11E−02 |
| EFS-6072 | 10.06 | 10.92 | 2.104 | 0.0201 | 9.55E−03 |
| EFS-6263 | 14.74 | 14.71 | 3.921 | 0.207 | 5.28E−02 |
| EFS-6461 | 30.49 | 22.21 | 7.114 | 0.183 | 2.57E−02 |
| EFS-6486 | 41.88 | 28.46 | 8.972 | 0.0776 | 8.65E−03 |
| EFS-6488 | 11.12 | 10.12 | 8.759 | 0.125 | 1.43E−02 |
| EFS-6488 | 13.53 | 12.87 | 8.759 | 0.125 | 1.43E−02 |
| EFS-6507 | 19.48 | 8.96 | 6.196 | 0.0344 | 5.55E−03 |
| EFS-6525 | 13.29 | 9.78 | 7.012 | 0.401 | 5.72E−02 |
| EFS-671- | 10.93 | 10.11 | 6.048 | 0.0511 | 8.45E−03 |
| EFS-6779 | 10.47 | 8.47 | 8.085 | 0.0654 | 8.09E−03 |
| EFS-6875 | 6.74 | 5.79 | 6.02 | 0.527 | 8.75E−02 |
| EFS-6891 | 15.42 | 8.53 | 8.272 | 0.338 | 4.09E−02 |
| EFS-7039 | 10.11 | 14.4 | 4.742 | 0.267 | 5.63E−02 |
| EFS-7055 | 9.58 | 9.42 | 5.88 | 0.219 | 3.72E−02 |
| EFS-7493 | 11.65 | 11.79 | 5.578 | 0.322 | 5.77E−02 |
| EFS-7506 | 13.09 | 16.17 | 3.447 | 0.309 | 8.96E−02 |
| EFS-7514 | 19.06 | 34.99 | 6.384 | 0.548 | 8.58E−02 |
| EFS-7755 | 9.15 | 11.17 | 8.559 | 0.347 | 4.05E−02 |
| EFS-7763 | 16.48 | 14.66 | 9.604 | 0.0391 | 4.07E−03 |
| EFS-7800 | 17.21 | 10.58 | 8.801 | 0.0119 | 1.35E−03 |
| EFS-7835 | 16.8 | 7.84 | 7.317 | 0.193 | 2.64E−02 |
| EFS-7851 | 18.27 | 14.79 | 7.243 | 0.0597 | 8.24E−03 |
| EFS-786- | 21.71 | 33.57 | 5.02 | 0.0649 | 1.29E−02 |
| EFS-7878 | 10.87 | 11.17 | 7.652 | 0.136 | 1.78E−02 |
| EFS-7886 | 16.22 | 15.84 | 5.224 | 0.0567 | 1.09E−02 |
| EFS-8993 | 17.32 | 15.71 | 9.099 | 0.614 | 6.75E−02 |
| EFS-9013 | 16 | 7.31 | 8.712 | 0.335 | 3.85E−02 |
| EFS-902 | 24.05 | 9.86 | 5.737 | 0.0228 | 3.97E−03 |
| EFS-9026 | 16.98 | 21.34 | 5.52 | 0.0584 | 1.06E−02 |
| EFS-9114 | 8.62 | 11.15 | 2.854 | 1.15 | 4.03E−01 |
| EFS-9224 | 14.53 | 21.22 | 9.459 | 0.0937 | 9.91E−03 |
| EFS-9291 | 15.98 | 10.83 | 9.887 | 0.143 | 1.45E−02 |
| EFS-9929 | 12.94 | 7.8 | 6.574 | 0.13 | 1.98E−02 |
| AK | 17.4 | 17.3 | 5.3 | 0.055 | 1.04E−02 |
| AS | 6.8 | 34.1 | 6.3 | 0.067 | 1.06E−02 |
| AT | 28 | 42.7 | 18.6 | 0.049 | 2.63E−03 |
| BP | 14.8 | 27 | 7.3 | 0.167 | 2.29E−02 |
| TM | 14.5 | 8.8 | 8.8 | 0.063 | 7.16E−03 |

Table 9: Values of the level of NE, MPO, cir-nDNA, cir-mtDNA, and MNR in non-severe, severe COVID-19 patients and healthy individuals. NS, non-severe (Table 9A, N=26); S, severe (Table 9B, N=44) COVID-19 patients and HI, healthy individuals (Table 9C, N=119).

### EXAMPLE

### Material & Methods

### Patients and Heathy Individuals

Blood samples from 114 healthy individuals were obtained from healthy donors, from the Etablissement Français du Sang (E.F.S), which is Montpellier's blood transfusion center (Convention EFS-PM N° 21PLER2015-0013). These samples were analyzed (virology, serology, immunology, blood numeration) and ruled out whenever any abnormality was detected.

Plasma samples from 32 patients with COVID-19 were provided by the CHU hospital of Montpellier (Centre Hospitalier Universitaire de Montpellier, France). Plasma samples from 10 individuals with colorectal cancer were obtained from the the ongoing UCGI 28 PANIRINOX study (NCT02980510/EudraCT n°2016-001490-33). Plasma samples from 10 patients with systemic lupus erythematosus (SLE) were provided by Dr. Perikles Simon from the Department of Sports Medicine, Prevention and Rehabilitation of Johannes Gutenberg University (Mainz, Germany).

### Samples Preparation

Blood from mCRC patients (n=219) were collected in STRECK tubes (Cell-Free DNA BCT®) and were sent within 24 hours of blood collection at room temperature from the recruiting institutions to our laboratory (IRCM, Institut de Recherche en Cancérologie de Montpellier, U1194 INSERM). Blood tubes were centrifuged for 10 minutes at 1,200×g at 4° C. within 5 days of blood collection, and the plasma supernatants were immediately centrifuged at 16,000×g at 4° C. for 10 minutes. Then, plasma samples were stored at −20° C. for several days or used immediately. Total circulating cell-free DNA was extracted from 1 mL of plasma using the QIAamp DNA Mini Blood Kit (Qiagen) in accordance with pre-analytic guidelines we have previously described (El Messaoudi, 2013; Meddeb, 2019) in an elution volume of 130 μL. CirDNA extracts were kept at −20° C. until use or used immediately. Blood from healthy individuals (n=114) was collected in EDTA tubes and was centrifuged for 10 minutes at 1,200×g at 4° C. within 4 hours of blood collection. Then, plasma supernatants were immediately centrifuged at 16,000×g at 4° C. for 10 minutes. Finally, plasma samples were stored at −20° C. for several days or used immediately.

### Quantification of cirDNA

Analysis of cirDNA was done by IntPlex®, an allele-specific blocker quantitative PCR (ASB Q-PCR), which we have described previously (Thierry et al., 2014; Mouliere et al., 2014), according to the MIQE guidelines (Bustin et al., 2009, 2010). Q-PCR amplifications were carried out in at least two replicates in a total volume of 25 μL on a CFX96 instrument using the CFX manager software (Bio-Rad). Each PCR reaction was composed of 12.5 μL of IQ Supermix Sybr Green (Bio-Rad), 2.5 μL of DNase-free water (Qiagen), 2.5 μL of forward and reverse primers (0.3 pmol/mL), and 5 μL of template. Thermal cycling comprised three repeated steps: a hot-start activation step at 95° C. for 3 minutes, followed by 40 cycles of denaturation-amplification at 95° C. for 10 seconds, then at 60° C. for 30 seconds. Melting curves were investigated by increasing the temperature from 60° C. to 90° C. with a plate reading every 0.2° C. Standard curves were performed for each run with a genomic extract of the DiFi cell line at 1.8 ng/μL of DNA. Each PCR run was carried out with no template control for each primer sct. Validation of Q-PCR amplification was performed by melt curve differentiation. Quantification of cirDNA concentration in mCRC patients and HI was obtained by amplifying a 67 bp-length wild-type sequence of the KRAS gene. In addition to routinely performing a standard curve for each primer couple with the PCR system, the accuracy and gene copy number variations were checked by quantifying a WT sequence of the BRAF gene from the amplification of a 90 bp amplicon. This method of quantifying cirDNA has been experimentally (MolOncol) and clinically validated (Nat Med, Annal oncol Thierry), and showed unprecedented specificity and sensitivity, to the point of permittingthe detection of a single DNA fragment molecule under Poisson Law distribution (AnnalOncol Thierry). An intra-and inter-experimental reproducibility study shows a 19% and 24% coefficient of variation (MolOncol, TransOncol) when jointly taking into consideration plasma preparation, cirDNA extraction and Q-PCR measurement.

### Human Myeloperoxidase (MPO) and Neutrophil Elastase (NE) Quantifications

MPO and NE concentrations were measured using enzyme-linked immunosorbent assay (ELISA) according to the manufacturer's standard protocol (Duoset R&D Systems, DY008, DY3174, and DY9167-05). Briefly, captured antibodies were diluted at the working concentrations in the Reagent Diluent (RD) provided on ancillary reagent kits (DY008) and coated overnight at room temperature (RT) on 96-well microplates with 100 μL per wells. Then, captured antibodies were removed from the microplate, and wells were washed three times with 300 μL of Wash Buffer (WB). Microplates were blocked at RT for 2 hours by adding 300 μL of RD to each well. RD were removed from the microplates, and wells were washed three times with 300 μL of WB. Then, 100 μL of negative controls, standards and plasma samples (diluted 1/10) were added to the appropriate wells for one hour at RT. Samples, controls and standards were removed from the microplates, and wells were washed three times with 300 μL of WB. Detection antibodies were diluted at the working concentrations in the RD, and then added by 100 μL per well, for one hour at RT. Detection antibodies were removed from the microplates, and wells were washed three times with 300 μL of WB. Then, 100 μL of Streptavidin-HRP was added to each well and microplates were incubated at RT for 30 minutes. Repeat wash three times. Finally, 100 μL per well of substrate solution was added and incubated for 15 minutes, and the Optical Density (O.D) of each well was read immediately at 450 nm with the PHERAstar FS instrument using the PHERAstar control software.

### Autoantibodies Anti-Cardiolipin (ACL) Antibody Index Calculation

The antibody index (AI) of total human autoantibodies against cardiolipin (IgG, IgM and IgA) was measured using direct ELISA according to the manufacturer's standard protocol (Boster, EK7027). Briefly, 100 μL of negative controls, positive controls, calibrator and diluted plasma samples ( 1/21) was dispensed into cardiolipin-coated wells and incubated for 30 minutes at RT. Samples, controls and calibrator were removed from the microplates and the wells were washed three times with 300 μL of WB. Then, 100 μL of enzyme conjugate was added in each well for 20 minutes at RT. The washing step was repeated. 100 μL of TMB substrate was dispensed into wells for 10 minutes. Finally, 100 μL of stop solution was added to each well and O.D was immediately read at 450 nm with the PHERAstar FS instrument using the PHERAstar control software. The cut-off value of each plate was calculated as follows: Calibrator O.D x Calibrator Factor (CF) of the kit. The antibody Index of samples was calculated by dividing the O.D of each samples by the cut-off value. We considered as positive for a NET derived inflammatory process, an individual with plasma sample with a cut-off/threshold value of 1.5 or 2 as determined by the ratio of the test value over the value of reference. The value of reference corresponds to the ACL value of one or more normal/healthy subject blood. Here, the reference value was determined from three normal/healthy subject blood samples.

### Statistical Analyses

The Mann-Whitney U test was used for non-parametric data. Correlation analysis was performed using the Pearson test (Graph Pad Prism 8.3.1 software). A probability of less than 0.05 was considered to be statistically significant; *p<0.05, **p<0.01; ***p<0.001; ****p<0.0001.

### Results

### Exemple 1: Human Plasma Isolation, Circulating cfDNA Extraction and Measurements in COVID-19 and Healthy Individuals

Human plasma isolation, circulating cfDNA extraction and measurement were performed on four COVID-19 patients being in critical care (N=18) and hospitalized (N=14) at time of blood draw as well on 113 healthy volunteers: All methods will be performed according to the pre-analytical guidelines previously established by our group (26): Blood collection in EDTA tubes; plasma isolation, double centrifugation; extraction by Qiagen Blood Mini Kit (Qiagen, CA), according to the manufacturer's protocol. Specific primers will be used to selectively amplify human DNA sequences of nuclear and mitochondrial origins as previously described (27,28). Importantly, targeted nuclear sequences generated amplicons of size lower than 80 bp. Circulating DNA (cirDNA) analysis followed: (i), our guidelines for pre-analytics; (ii), IntPlex methodology for quantification (27); (iii), DNA fragmentation will be evaluated by calculating the DNA Integrity Index (DII) (29); (iv), quantification of mitochondrial cirDNA; and (v), mitochondrial to nuclear cirDNA ratio (MNR). CirDNA concentration are determined by Q-PCR analysis in triplicate and are expressed as ng/ml (Table 1 and 2).

The COVID-19 patients are discriminated into patients tested as hospitalized (N=14) and as in ICU (Intensive Care Unit, N=18). The healthy individual cohort is composed of 113 subjects.

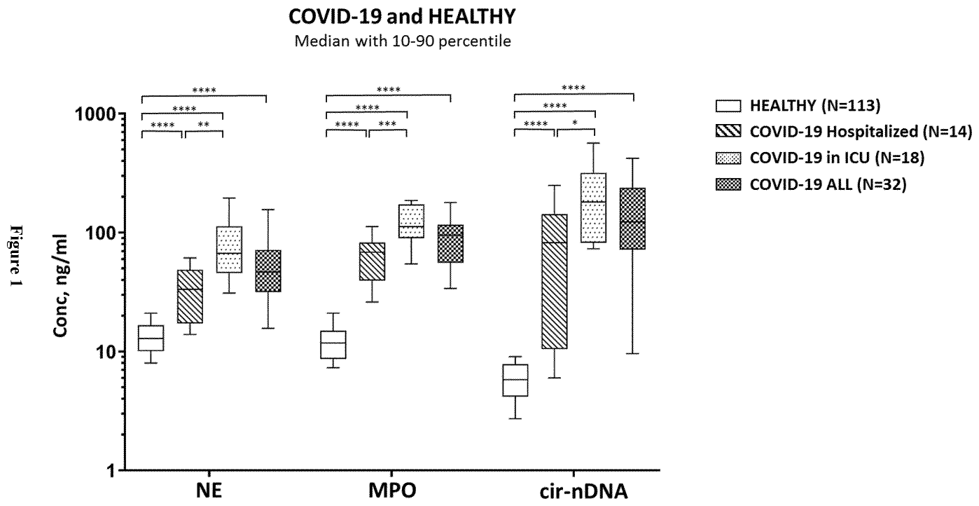

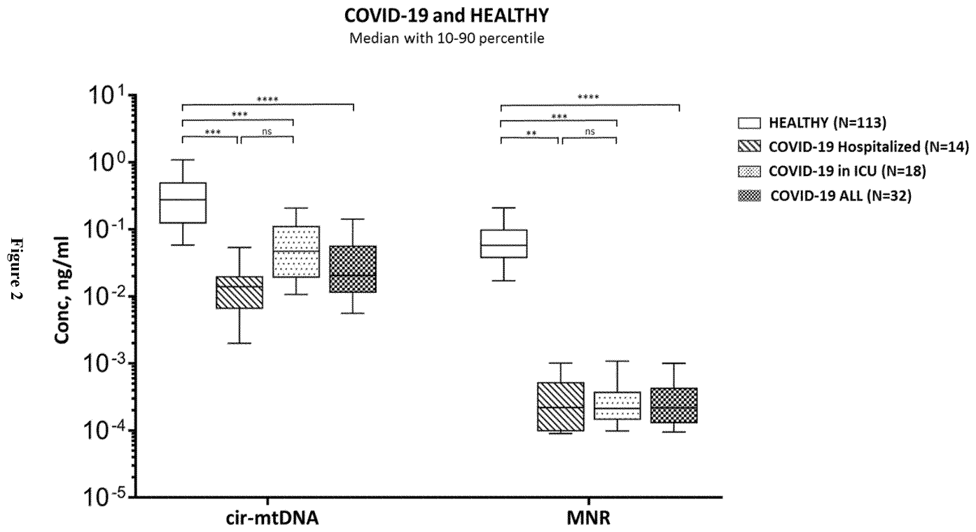

Data are presented in FIGS. 1 and 2, and Table 1. Altogether, we observed that:
Nuclear cirDNA concentration (cir-nDNA) values are on the average of 16-fold higher in COVID-19 hospitalized than in healthy subjects (82.1+/−86.2 vs 5.8+/−2.3 ng/ml); p<0.0001.
Nuclear cirDNA concentration (cir-nDNA) values are on the average of 43-fold higher in COVID-19 ICU than in healthy subjects (149.02+/−61.4 ng/mL vs 3.6+/−0.2 ng/ml); p<0.000001.
Mitochondrial cirDNA concentration (Cir-mtDNA) values are on the average of 20-fold lower in COVID-19 hospitalized than in healthy subjects (0.014+/−0.017 ng/mL vs 0.277+/−0.373 ng/mL).
Mitochondrial cirDNA concentration (Cir-mtDNA) values are on the average of 7-fold lower in COVID-19 ICU than in healthy subjects (0.042+/−0.123 ng/mL vs 0.277+/−0.0373 ng/ml).
The MNR (cir-mtDNA over cir-nDNA concentration ratio) is on the average of 150-fold higher in both COVID-19 hospitalized and ICU than in healthy subjects (0.0002 and 0.0002 vs 0.05).

Our data revealed a high significant statistical difference between COVID and healthy individuals. Thus, cir-nDNA and cir-mtDNA are independent biomarkers for COVID-19. The MNR is, also, a potential strong biomarker for COVID-19.

### Exemple 2: Quantification of MPO and NE in COVID-19 and Healthy Individuals

Quantification of MPO and NE: MPO (myeloperoxidase) and NE (neutrophil elastase) will be measured using ELISA according to the manufacturer's standard protocol (Duoset R&D Systems, DY3174, and DY9167-05). Data are presented in FIG. 1 and Table 2. Altogether, we observed that:
MPO values are on the average of 6-fold higher in COVID-19 hospitalized than in healthy subjects (68.3+/−30 ng/mL vs 11.8+/−9.5 ng/ml), p<0.0001.
Elastase values are on the average of 3-fold higher in COVID-19 hospitalized than in healthy subjects (33.3+/−17.2 ng/mL vs 12.9+/−8.3 ng/ml), p<0.0001.
MPO values are on the average of 12-fold higher in COVID-19 ICU than in healthy subjects (122.3+/−47.4 ng/mL vs 11.8+/−9.5 ng/ml), p<0.000001.
Elastase values are on the average of 6-fold higher in COVID-19 ICU than in healthy subjects (75.3+/−59.2 ng/mL vs 12.9+/−8.3 ng/mL), p<0.000001.

Our data revealed very high significant statistical difference between COVID and healthy individuals (Table 3). Thus, MPO and NE concentration are independent biomarkers for COVID-19.

When combining these observations with those in example 1, we can state that (i), there is no overlap in MPO, NE, or cir-nDNA (FIG. 1) measurements when values from individual patients within both healthy and COVID ICU groups are compared; and (ii), there is no significant overlap in measurements when values from individual patients within both healthy and COVID hospitalized groups are compared.

There is no overlap in cir-mtDNA (FIG. 2) measurements when values from individual patients within both healthy and COVID ICU groups are compared.

There is no overlap in MNR (FIG. 2) measurements when values from individual patients within both healthy and COVID groups are compared.

Altogether, COVID-19 hospitalized and in ICU patients showed as compared to the healthy group a 3- and a 6-fold increase, a 6- and a 12-fold increase, 16- and a 43-fold increase, 20- and a 7-fold decrease, and equivalent values in respect to NE, MPO, cir-nDNA, cir-mtDNA and the MNR, respectively (Table 4). As a consequence, there is a gradual increase of NE, MPO and cir-nDNA from Healthy, to hospitalized and ICU patients; and a gradual decrease of cir-mtDNA from hospitalized to ICU patients. A very high statistical difference (a decrease of 150-fold) was observed for both hospitalized and ICU patients as compared to the healthy group (Table 4).

Suggested COVID-19 positive threshold can be inferred for these data such as a 2-fold increase for NE MPO and cir-nDNA; a 5-fold decrease for cir-mtDNA; and a 10-fold decrease for the MNR.

### Exemple 3: Analysis of the Correlation of the NET Biomarkers in COVID-19 and Healthy Individuals

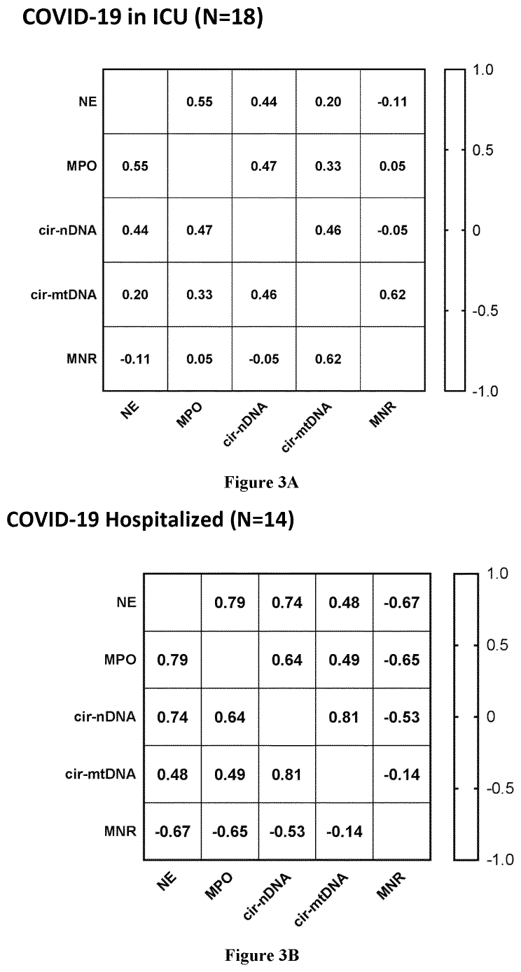

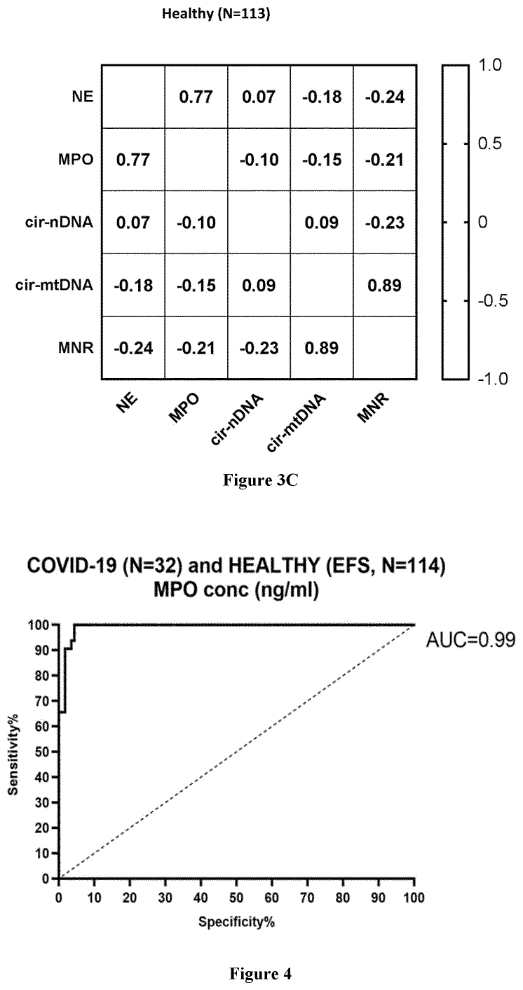

Pearson correlation were performed to show correlation of MPO with cir-mtDNA and of NE with cir-nDNA (FIG. 3), both when comparing with level obtained in healthy and COVID-19 patients. Correlation values are r values and are indicated in correlation square presentation (FIG. 3)

Data are presented in FIG. 3. Overall, we observed that:
Among MPO, NE, cir-nDNA, and cir-mtDNA, only MPO and Elastase correlated in Healthy subjects (high correlation value: 0.77).
MPO, NE, cir-nDNA, and cir-mtDNA values correlated in all COVID patients.
Elastase and cir-nDNA correlated in COVID hospitalized and ICU subjects while no or inverse correlation is seen in Healthy subjects.
MPO and cir-nDNA correlated in COVID hospitalized and ICU subjects while poor or no inverse correlation is seen in Healthy subjects.
Cir-nDNA and cir-mtDNA correlated in COVID hospitalized and ICU subjects, whereas no correlation exist in healthy subjects.
Elastase and cir-mtDNA slightly inversely correlated in Healthy subjects while no and a poor correlation is seen in COVID hospitalized and ICU subjects, respectively.
MPO and cir-mtDNA slightly inversely correlated in Healthy subjects while a poor and a positive correlation is seen in COVID hospitalized and ICU subjects, respectively.

Respective values of the NET biomarkers differently correlate in healthy and COVID-19 individuals. Thus, these differential correlations between these biomarkers are parameters flagging of COVID-19.

In addition, the positive correlation of cirDNA level with NET biomarkers such as NE or MPO is specific to covid patients and can be considered as a biomarker of the COVID pathology.

### Exemple 4: Determination of Correlation Indexes in COVID-19 and Healthy Individuals

Calculation of indexes based on the differential correlations between correlation of NET markers for characterizing COVID-19 or inflammatory pathologies.

We associated NETs markers (NE, MPO, Cir-mtDNA and Cir-nDNA) that are determined in each tested individuals, and correlation indexes associating two or three markers are calculated (Table 5 and 6). Data clearly showed that some ratios of two markers and correlation indexes of ratios combining the three markers revealed power in discriminating the group of COVID-19 patients to the group of healthy individuals. The difference is only statistically significant only for NE/Cir-mtDNA, MPO/cir-mtDNA and NE x MPO/Cir-mtDNA correlation indexes. These correlation indexes or ratio reveal a specific characteristic for patient with COVID-19 and are powerful candidates for diagnosing or monitoring COVID-19. The diagnostic power is an arbitrary unit considering the presence of overlap values and median statistical difference. Thus, NE/Cir-mtDNA, MPO/cir-mtDNA and NE x MPO/Cir-mtDNA showed a moderate, intermediate and high diagnostic power.

### Exemple 5: Cir-mtDNA is a Marker Useful for Diagnosing Inflammatory Diseases, Such as COVID-19; Use of the Ratio of the Concentration of cir-mtDNA Over the Concentration of cir-nDNA

We compared the respective concentration of cir-mtDNA and the concentration of cir-nDNA by calculating the MNR (Table 1 and 2, and FIG. 2). Pearson correlations were performed to show the correlation of the MNR with NE, MPO, cir-nDNA and cir-mtDNA (FIG. 3).
The MNR only correlated with the cir-mtDNA in healthy and ICU subjects, while it does not in the hospitalized patient group.
The MNR inversely correlated with NE, MPO, and cir-nDNA in healthy and hospitalized subjects.
The MNR poorly correlated with NE, MPO, and cir-nDNA in ICU subjects.

The study of correlation of the MNR values enables to distinguish hospitalized to ICU patients. This property is original and certainly of high interest since blood markers of the severe disease are rare and critical in COVID-19 patient monitoring.

### Example 6: Cir-mtDNA Detection as a Platelet Activation Marker

We previously proved that the detected cir-mtDNA correspond mainly to circulating cell-free extra-cellular mitochondria (Al Amir Dache, 2020). Comparing values of cir-mtDNA content from plasma prepared in only the 1200 g first centrifugation to that of the 16,000 second centrifugation step correspond to the number of cell-free mitochondria. In addition, it is established in the literature that platelets release cell-free mitochondria upon activation. Since COVID-19 is associated to platelets activation, we propose here for the first time that the ratio or the respective proportion of the cir-mtDNA content as determined from the conventional plasma preparation (first low speed centrifugation 300-1500 g for at least 10 minutes) and the plasma prepared following a second (high speed) centrifugation (at least 10,000 g for at least 10 minutes) is associated to platelet activation and considered as a biomarker of inflammatory diseases, such as covid-19.

Thus, as observed in Table 1, the ratio of cir-mtDNA median concentration as determined at low-speed centrifugation (1200 g) over the cir-mtDNA median concentration as determined at high-speed centrifugation (16,000 g) is ˜40- (1.9 vs 0.047 ng/ml) and ˜100-fold (1.6 vs 0.014 ng/ml) higher in ICU and hospitalized patients, while being only of ˜3-fold (0.82 vs 0.277 ng/ml). (Table 1)

Note, the ratio of the cir-mtDNA content over cir-nDNA content (we previously called MNR in studies on cancer screening test) as determined at low speed centrifugation (1200 g) over the median MNR as determined at high speed centrifugation (16,000 g) is ˜36- (6.01×10³ vs 2.13×10⁴) and ˜100-fold (1.6×10² vs 2.2×10⁴) higher in ICU and hospitalized patients, while being only of ˜3-fold (1.42×10¹ vs 5.8×10²) (Table 2). This confirms the robustness of the previous marker (the respective proportion of cir-mtDNA median concentration as determined at low speed centrifugation as compared to the cir-mtDNA median concentration as determined at high speed centrifugation).

### Example 7: The Degree of cir-nDNA Fragmentation is a Marker of an Inflammatory Disease, such as COVID-19

As previously reported in cancer pathology, we calculated the DNA Integrity index (DII) (Table 1). It is determined as the ratio of long over short fragment content, by analyzing by Q-PCR the quantity of amplicons generated by targeting here a 67 bp and 305 bp sequence in the monogenic KRAS gene. As presented in Table 1, the median DII is 0.105, 0.039, and 0.056 in healthy, hospitalized and ICU subjects, respectively. This revealed a higher cir-nDNA fragmentation in COVID-19 patients, suggesting a capacity of distinguishing healthy to COVID-19 subjects.

CirDNA fragment profile can be determined precisely by LP-WGS. As shown in the FIG. 10, cancer cirDNA size profile shows a slight but reliable shift to the lower size a peak at at 167-168 bp. Several parameters as determined from the size profile enable to characterize mCRC and COVID-19 patients and enable the screening of cancer patients.

### Example 8: NE, MPO, cir-nDNA, cir-mtDNA and MNR are Powerful Markers Enabling to Discriminate an Inflammatory Disease, Such as COVID-19, and Useful for COVID-19 Patient Screening

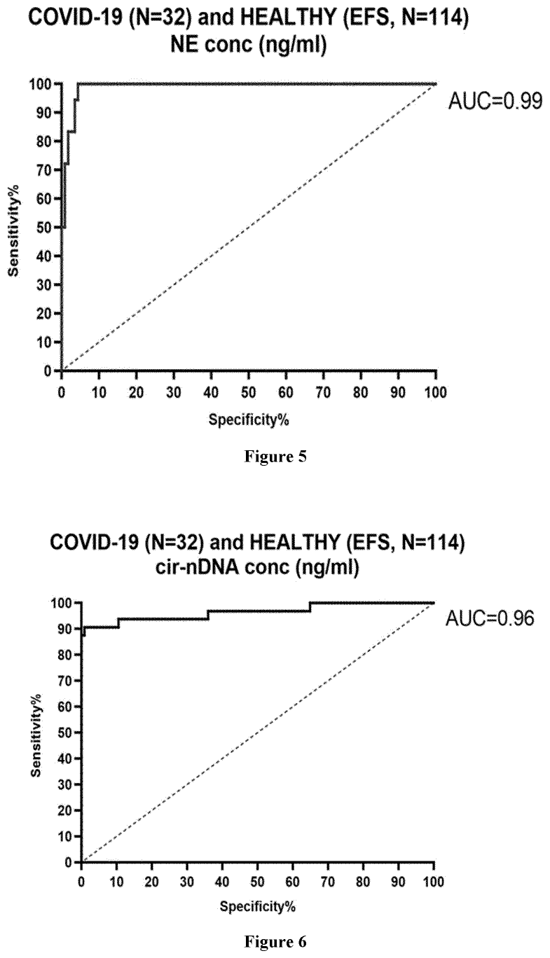

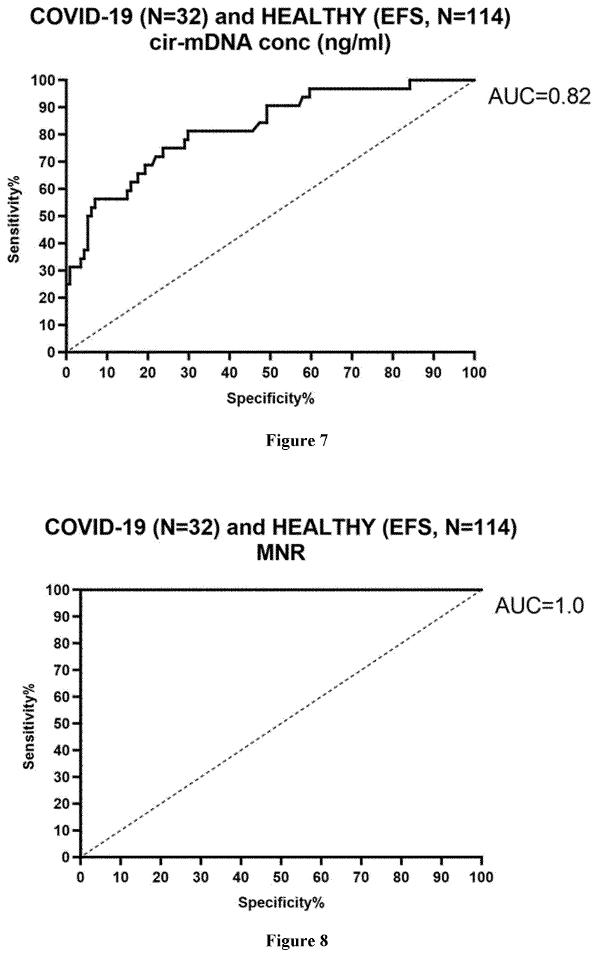

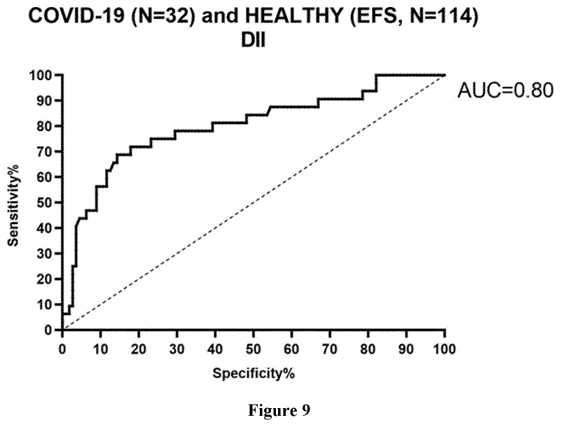

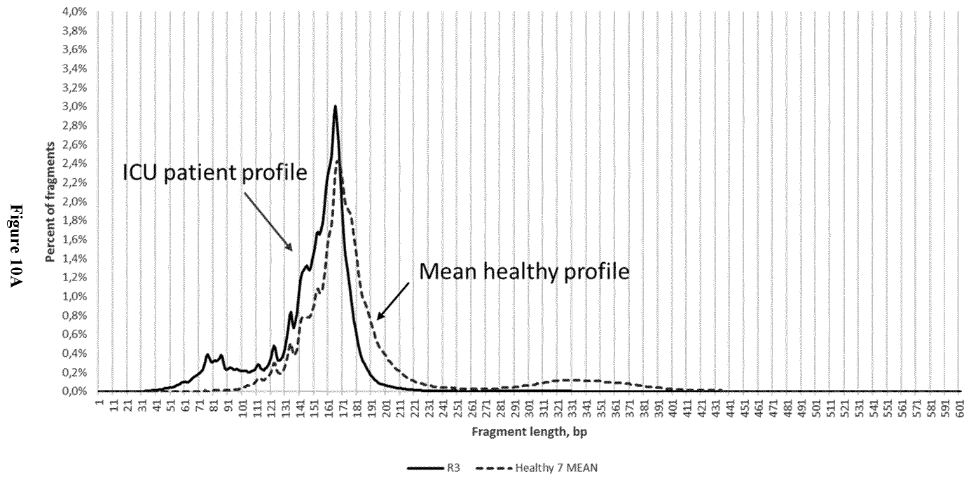

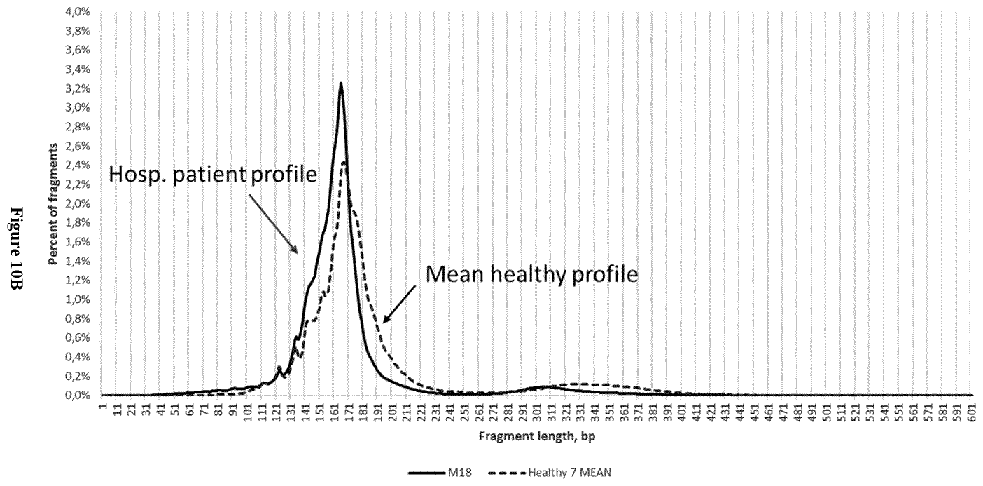

FIGS. 4-9 show ROC curves analysis of the data previously obtained in the 32 COVID-19 patients and in the 113 healthy individuals. Very high AUC values were calculated for NE, MPO, cir-nDNA, cir-mtDNA and MNR, and to a lesser extent DII (0.99, 0.99, 0.96, 0.82, 1.0, and 0.80). These AUC values are unmatched in the literature, and clearly demonstrated that these markers are powerful to screen COVID-19 patients to healthy individuals. This suggest that the association of these markers in a combined test would be not only a strong biomarker for the patient follow up but also a strong screening test for COVID-19, and inflammatory diseases.

### Exemple 9: NE, MPO, cir-nDNA, cir-mtDNA and MNR are Markers for Cancer, Such as Metastatic Colorectal Cancer (mCRC)

The concentration of NE, MPO, and cir-nDNA, in 10 mCRC patients and in 113 healthy subjects (Table 7) were represented as histograms in the FIG. 11. Mean values in mCRC patients are clearly much higher than those determined in healthy individuals and are statistically differents (Table 8).

### Exemple 10: Correlation of NE, MPO, cir-nDNA, in Cancer, Such as Metastatic Colorectal Cancer (mCRC)

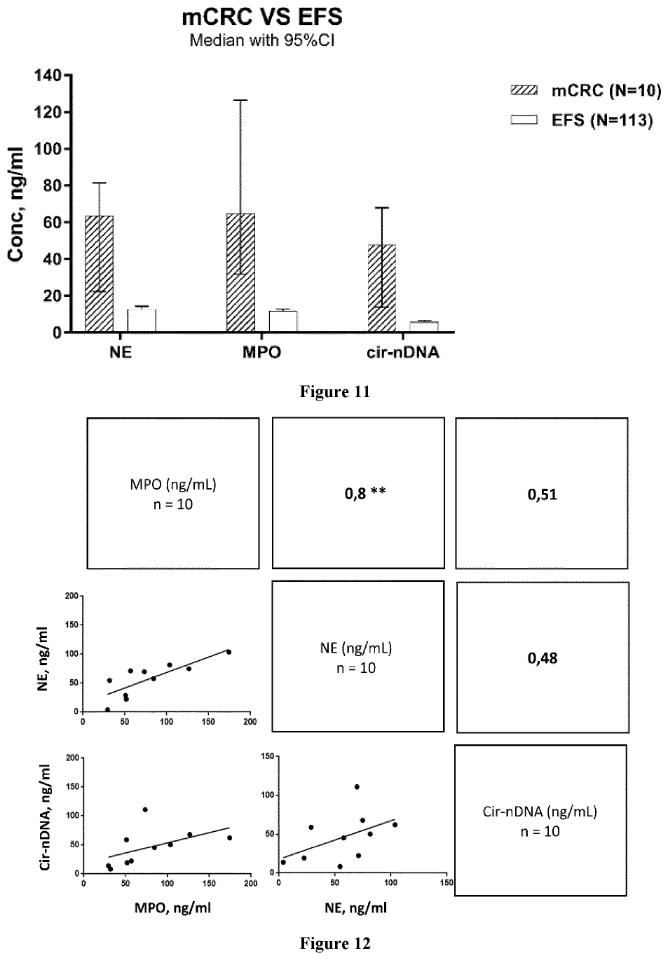

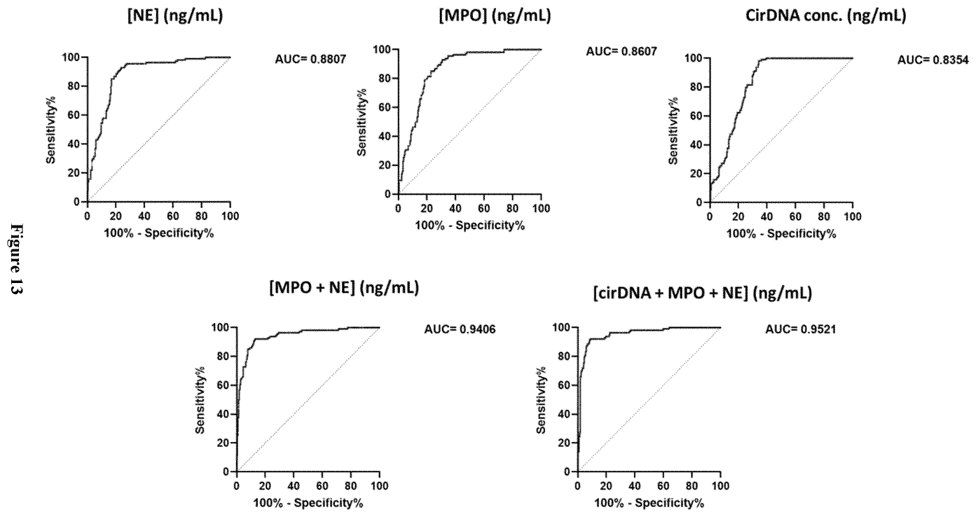

Pearson correlation analysis of NE, MPO, and cir-nDNA concentration values as previously determined (Table 7) is shown in FIG. 12. High correlation indexes (0.8, 0.51 and 0.48) were calculated between MPO and NE, MPO and cir-nDNA, and NE and cir-nDNA, respectively. Cir-nDNA correlation with NE and MPO was absent in healthy individuals (FIG. 3). This buttress the notion that NE, MPO, and cir-nDNA are intimately associated in mCRC, strongly suggest that these compounds are by-products of the NETs, and confirm as in COVID-19 patient plasma that cancer derived cir-nDNA largely originate from the NET degradation in mCRC.

### Example 11: NE, MPO, and cir-nDNA, are Powerful Markers Enabling to Discriminate an Inflammatory Disease, Such as Cancer, and Useful for Cancer Patient Screening

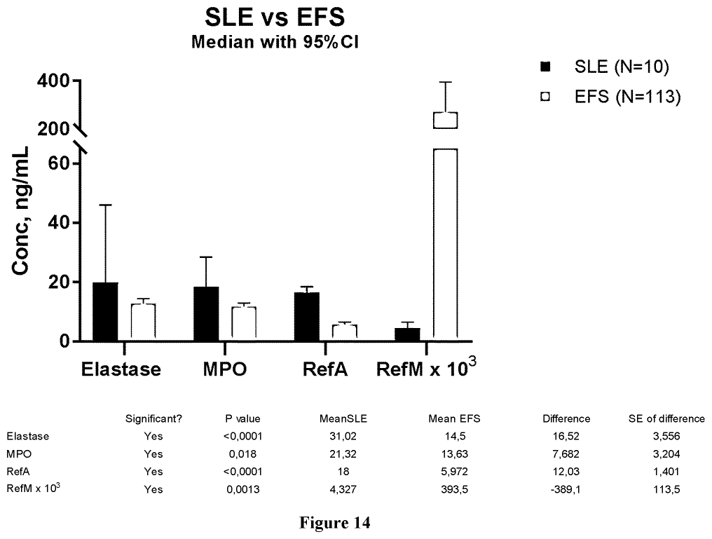

FIGS. 13 shows ROC curves analysis of the data obtained in the 10 mCRC patients and in the 113 healthy individuals. High AUC values were calculated for NE, MPO, cir-nDNA, (0.88, 0.86, and 0.84, respectively). This clearly demonstrated that these markers are powerful to screen cancer patients from healthy individuals. This suggest that the association of these markers in a combined test would be not only a strong biomarker for the patient follow up but also a strong screening test for cancer. This observation extend that made for COVID-19 patients, highlighting their screening power for inflammatory diseases.

### Exemple 12: NE, MPO, cir-nDNA, cir-mtDNA and MNR are Markers for an Auto-Immune Disease, Such as SLE

The concentration of NE, MPO, cir-mtDNA and cir-nDNA, in 10 lupus patients and in 113 healthy subjects (Table 7) were represented as histograms in the FIG. 14. Mean values in mCRC patients are clearly much higher than those determined in healthy individuals and are statistically differents (FIG. 14).

### Exemple 13: Correlation of NE, MPO, cir-nDNA, in an Auto-Immune Disease, Such as SLE

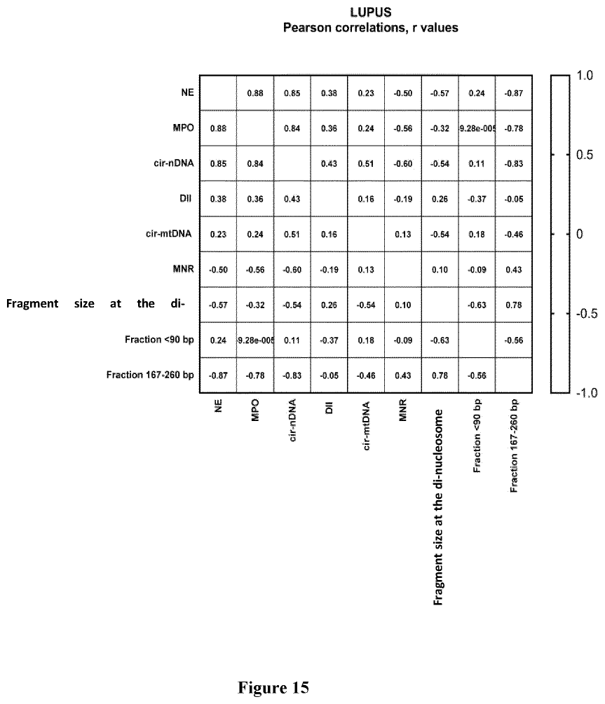

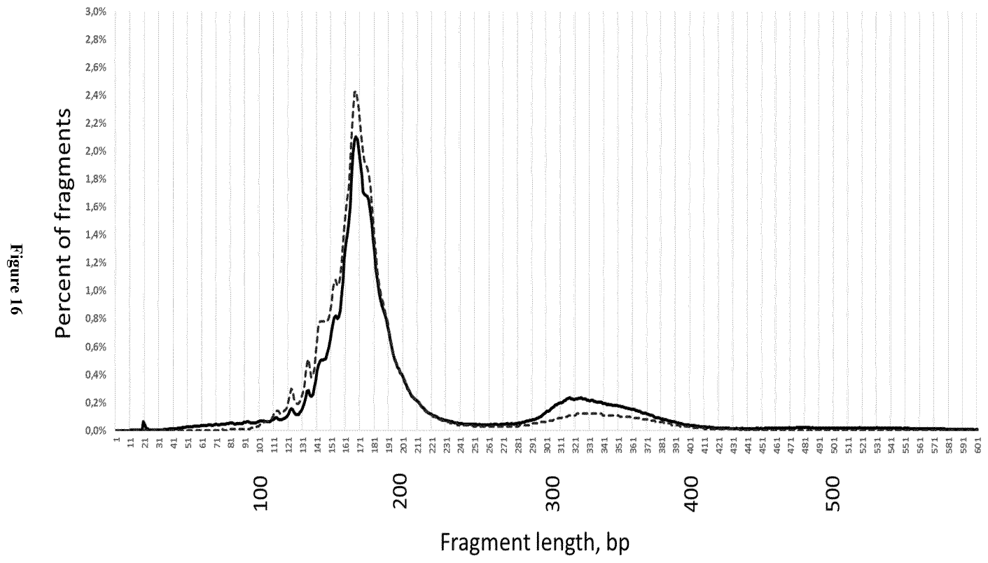

Pearson correlation analysis of NE, MPO, cir-nDNA, cir-mtDNA concentration and the MNR and DII values as previously determined (FIG. 14) is shown in FIG. 15. High correlation indexes (0.88, 0.85 and 0.85) were calculated between MPO and NE, MPO and cir-nDNA, and NE and cir-nDNA, respectively. Cir-nDNA correlation with NE and MPO was absent in healthy individuals (FIG. 3). Note, the MNR strongly and inversely correlates with NE, MPO, cir-nDNA. This buttress the notion that NE, MPO, cir-nDNA cir-mtDNA concentrations and the MNR and DII values are intimately associated in SLE, and strongly suggests that NE, MPO, and cir-nDNA are by-products of the NETs, and confirm as in COVID-19 and mCRC patient plasma that cir-nDNA largely originate from the NET degradation in SLE. As for COVID-19 and mCRC, cir-nDNA from lupus show a fragment size profile difference (but in this case much slighter) with healthy subject plasma (FIG. 16). Altogether, the correlation studies with using COVID-19, SLE and mCRC as models show similar characteristics between those inflammatory diseases. We infer that NET by-products such as NE, MPO, cir-nDNA, cir-mtDNA) are markers that can be combined.

Note, SLE plasma cir-nDNA size profile appears to vary as compared to that of healthy individuals. The proportion of the fragments of range 30-90, 90-168 and 300-420 are slightly higher, lower and higher than that of healthy individuals (FIG. 16).

### Exemple 14: NE, MPO, cir-nDNA (cirDNA) and MNR are Characterizing Inflammatory Diseases

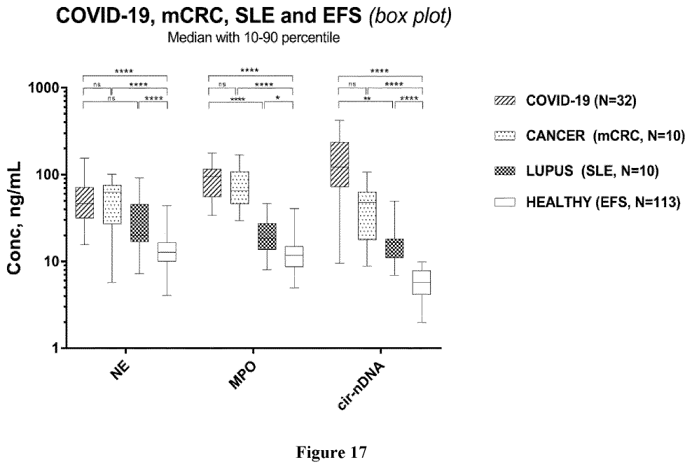

FIG. 17 combine all previous data presented in FIGS. 1, 11 and 14. NE, MPO, and cir-nDNA concentration values are statistically different as compared to those determined in healthy individuals. They appear to help in discriminating mCRC, COVID-19, and SLE patients from healthy (EFS) individuals. Altogether, the study of the quantitative levels and the correlation studies of plasma of patients with inflammatory diseases such as COVID-19, SLE and mCRC show similar characteristics. Despite their different nature, these disorders appear as inflammatory disease models. We infer that NET by-products such as NE, MPO, cir-nDNA, and cir-mtDNA) are markers that can be used to diagnose and to follow-up individuals with inflammatory diseases. The data clearly demonstrated their high diagnostic power that may be even improve when combining them.

### Exemple 15: Auto-Anticorps Cardiolipin (aCL) Levels in COVID-19 Patients

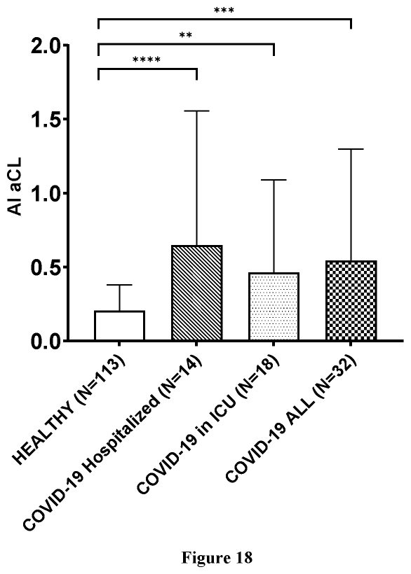

Auto-anticorps cardiolipin (aCL) appears elevated in COVID-19 patients as compared to healthy subjects (FIG. 18). ACL mean level in hospitalized, ICU and all COVID-19 patients is about 3-fold, 2-fold and 2.5-fold higher than the mean level in the healthy subjects (N=113). Statistical differences were observed between healthy individuals and each COVID-19 patient groups (hospitalized, ICU, or ICU+hospitalized) (FIG. 18). We considered a threshold value of 1.5 or 2 as determined by the ratio of the test value over the value of reference. The value of reference correspond to the ACL value of one or more normal/healthy subject blood. Here, the reference value was determined from three normal/healthy subject blood samples.

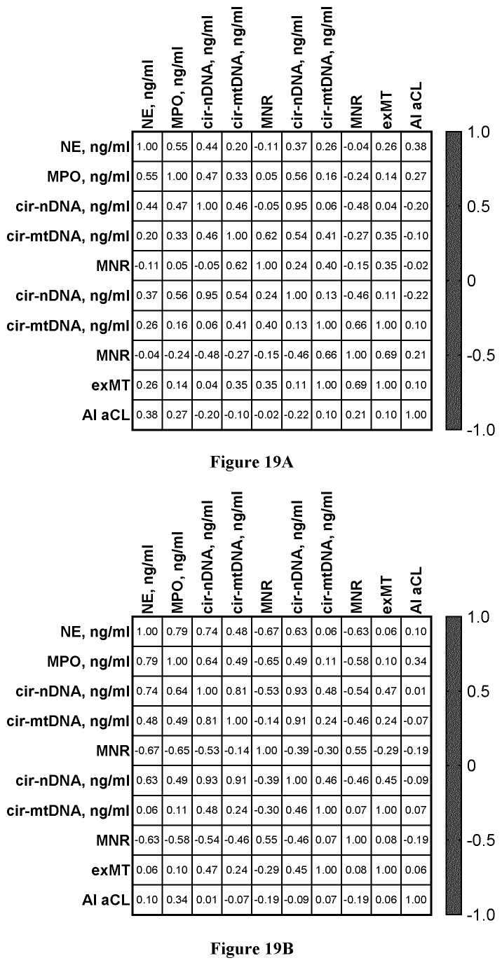

Pearson correlation values of aCL and the various other inflammatory parameters analysed in this invention are presented in FIG. 19. ACL levels only correlate with NE and MPO in ICU or hospitalized patients (FIG. 19).

### Example 16: Most of the mCRC Patient Plasma Show Presence of ACL Auto-Antibodies

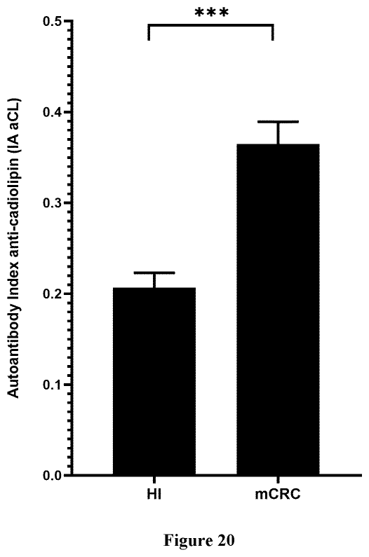

The mean concentration of ACL in the concentration index as determined by ELISA is 0.2+/−0.02 and 0.36+/−0.03 (+/−SD) in healthy (N=113) and mCRC plasmas (N=232) (FIG. 20). The difference is statistically significative (P<0.001).

### Example 17: Results Obtained on Long Covid-19 Patients

### Methods

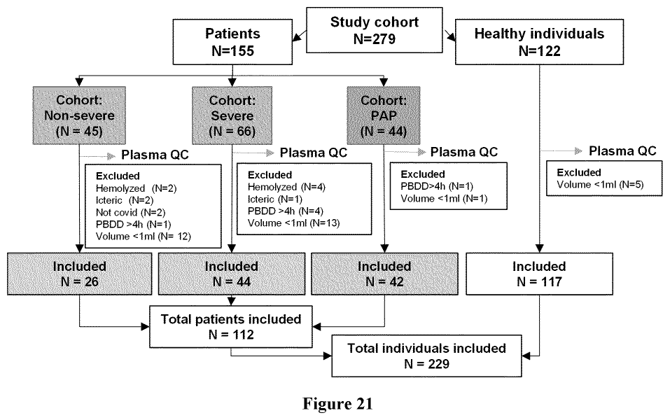

Among the 279 plasma from COVID-19 patients and HI individuals enrolled in the study, 229 (26 S (severe), 44 NS (non-severe), 42 PAP (post-acute phase), and 117 HI) passed the quality control step and were subsequently analyzed (FIG. 21). All the COVID-19 (NS and S) patients exhibited general COVID-19 symptoms and characteristics as reported elsewhere 1 (Supplement 1). We categorized patients as S vs NS depending on whether or not they met one or more of the following criteria: need for high flow nasal oxygen therapy (Optiflow; O2>15 L/min) or mechanical ventilation, transfer to the ICU during hospitalization, or occurrence of death. The group of PAP patients consisted of 42 subjects previously hospitalized in an ICU who were offered longitudinal monitoring 6 months or more after discharge, whether or not they were judged to have returned to full health.

### Results

We observed that NE, MPO and cir-nDNA concentrations in plasma were statistically significantly elevated in COVID-19 NS and S patients compared to HI (data not shown). The highest values of NETs markers were found in the plasma of severe COVID-19 patients, which showed significant differences from HI in the analysis of NE (74.6 ng/ml vs 12.9 ng/ml, p<0.000001), MPO (112.9 ng/ml vs 12.2 ng/ml, p<0.000001) and cirDNA (134.9 ng/ml vs 5.9 ng/ml, p<0.000001). The statistical differences found here between COVID patients and healthy subjects are higher than previously reported 5,8, 10. Values of these markers in S patient plasma are statistically higher than in NS patients (data not shown; p<0.0001 for both NE and MPO; and p<0.005 for cir-nDNA). In light of our previous studies (38), we also applied an index determined by the cir-mtDNA/cir-nDNA ratio (MNR), which demonstrates a high capacity to differentiate cirDNA according to its origin. In this study, cir-mtDNA, and MNR were significantly lower in COVID-19 S and NS patients compared to HI (data not shown). There was no correlation between cir-nDNA and NE/MPO concentrations in HI, while they correlated positively in COVID-19 patients (data not shown). Cir-mtDNA did not associate with cir-nDNA in HI, and correlate positively in S and NS patients. MNR did not correlate or correlated weakly and negatively with cir-nDNA, NE, and MPO in HI and NS patients, but did not correlate with NE and MPO in S and PAP patients. Note, the significant statistical MNR decrease we observed here in COVID-19 patients might suggest compromised mitochondria-nuclear co-regulation, as speculated by Medini et al (39).

Thus, our data confirmed observations we previously made in relation to metastatic colorectal cancer, namely that NETs protein biomarkers are associated with the generation of cirDNA, clearly demonstrating that NETs degradation in blood leads to chromatin fragmentation mostly resulting at the end to circulating mononucleosomes associated DNA. In addition, our present study confirmed both our own previous postulates and those of Barnes et al (2), which clearly link the production of NETs in COVID-19 patients and highlight the potential NETs key role in COVID-19 pathogenesis. In addition to the release of excessive amounts of pro-inflammatory cytokines, acute infection is associated with a high number of hyperactivated degranulating neutrophils. The by-products of NETs may be implicated in the pathogenesis of COVID-19, with elastase notably playing a role in accelerating virus entry. As in numerous other NETopathies, those by-products may also induce hypertension, thrombosis and vasculitis. We speculate that SARS-CoV2 may activate an innate immune response, resulting in an uncontrolled formation of NETs, and inducing multi-organ failure in high risk individuals.

We observed aCL and anti-B2GP presence in a significant fraction of NS and S patients (38.9% IgM and IgG, and IgM and IgG 23.1%, respectively). ACL correlation with anti-B2GP is clearly apparent (data not shown), as has previously been observed for various diseases, such as APS17. The aCL prevalence levels we determined correspond to those reported in several very recent reports on COVID-19 (40, 41 and 42). Associations between both antibodies as well as between NETs markers and both antibodies were observed in the S group, and to a lesser extent in the NS group (data not shown). Although the detection of aPL such as aCL has shown potential as a strategy in preventing thrombosis, the direct or indirect role of aPL in COVID-19 thrombophilic coagulopathy has yet to be fully understood. Shi et al (43) spectulate that endothelial cells may be activated by aPL, which may in turn induce a pro-adhesive phenotype. That said, the contribution of neutrophils and NETs to anti-phospholipid syndrome (APS) pathophysiology is nonetheless evident (44). The link has also been established between exacerbated NETs formation and APS in multiple auto-immune and non-auto-immune pathologies (including lupus) which exhibit raised aCL levels. Note, the progressive expansion of the intima by cell proliferation, leading to organ damage, characterizes occlusive vasculopathy in APS6. In addition, thrombotic complications have been reported to associate with aCL positivity in some cases of a variety of viral infections. NETs and thrombi were found to colocalize in COVID-199; more precisely, cirDNA and MPO activity were associated in patients with thrombotic micro-angiopathies.

While the concentrations of NE, MPO and cir-nDNA were lower in PAP patients as compared to NS and S, they were statistically higher than in HI (PAP vs HI: NE: 16.8 vs 12.9ng/μl; MPO: 25.7 vs 12.2 ng/μl; cir-nDNA: 15.2 vs 5.9 ng/μl). There was also a difference in the cir-mtDNA, and MNR values of HI and PAP subjects (data not shown). There was a clear correlation between cir-nDNA and NE/MPO concentrations in PAP patients, while MNR and cir-mtDNA were not associated with NE, MPO and cir-nDNA (data not shown). While their prevalence did not correlate with NETs markers, in contrast to S patients, aCL and aB2GP were detected in 19.1% of PAP patients (data not shown). Although the presence of these two auto-antibodies was clearly detected in several patients (7/26, 9/44, and 8/42 in NS, S and PAP respectively), with a significant prevalence, the fact that the positive patient number was rather low means that their prevalence value should nonetheless be treated with caution.

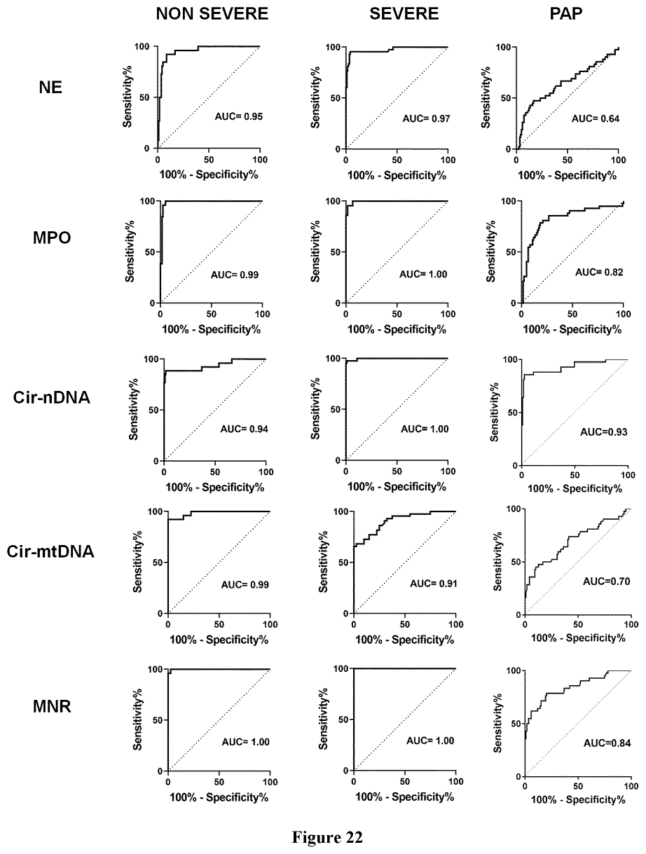

NETs and cirDNA markers showed high diagnostic capacity: As determined from receiver operating characteristics (ROC) curves (FIG. 22), NE (area under curve AUC 0.95, 0.97 and 0.64), MPO (0.99, 1.00, and 0.82) and cir-nDNA (0.94, 1.00, and 0.93) showed high levels of diagnostic capacity for NS, S and PAP individuals, respectively, as compared with HI. When comparing a combination of both NS and S COVID-19 patient cohorts with HI, we observed AUC of 0.97, 0.99, 0.98, and 1.0 for NE, MPO, cir-nDNA, and MNR, respectively (data not shown); when differentiating NS and S, we observed AUC of 0.81, 0.81, 0.72, and 0.60 for NE, MPO, cir-nDNA and MNR, respectively (data not shown). Note, cir-nDNA showed a higher diagnostic capacity (AUC of 0.93 as compared to 0.64 and 0.82 for NE and MPO, respectively) in the PAP “long covid” patients (FIG. 22). Thus, cir-nDNA may in some clinical conditions be of higher performance than conventional NETs proteic markers to diagnose inflammatory diseases.

This work is the first to reveal that NETs and aCL production may be sustained for 6 months or more post-acute infection. While the PAP subjects we studied were not categorized as “long COVID” patients, most nonetheless experienced mild prolonged COVID symptoms.

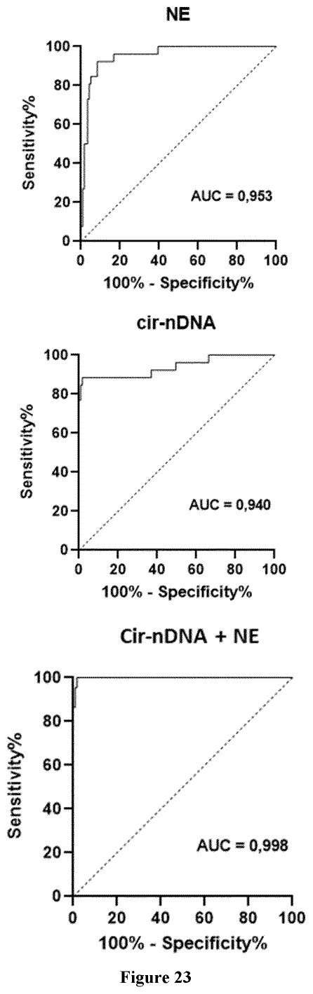

We speculate that uncontrolled NETosis activation resulting from SARS-CoV2 infection may be sustained by a feed-back loop resulting from systemic NETs byproducts release. Active investigation is urgently needed to understand the nature of this serious and long-lasting phenomenon, and then to develop suitable therapy towards complete recovery. The biomarkers examined in this work showed a very high diagnostic power (FIGS. 22 and 23), exhibited association with disease severity, a higher diagnostic performance when combined and may contribute significantly to achieving this public health objective.

### Example 18: Combining NETs and cirDNA Markers Showed Higher Diagnostic Capacity

CirDNA markers such as cir-nDNA or cir-mtDNA or MNR not only are new markers for NETs (4), but they are correlated with NETs proteic markers (FIG. 19) and their combination with NETs proteic markers using threshold values as presented in this invention (higher than 21, 21.5, 9 ng/ml and lower than 0.1 ng/ml and 0.002, for NE, MPO, cir-nDNA, cir-mtDNA and MNR, respectively) showed higher diagnostic capacity. As for numerous clinical diagnostic tests, it is safer to base diagnosis on multiple analytes, since erroneous results may arise from technical problems. Therefore, combining data from NE and/or MPO and/or cir-nDNA and/or cir-mtDNA and/or MNR provide higher diagnostic performance. The lesser the false positive and false negative rate, the more performant the diagnostic test is. Combining positive results for NETs and cirDNA markers improve AUC value and consequently test performance as illustrated in FIG. 23 and reduced the rate false positives as below described from data of the study presented in example 17 (Table 9).

FIG. 23 showed the ROC and AUC of the severe cohort of the NE values alone with concentration higher than 20 ng/mL, of the cir-nDNA values alone with concentration higher than 9 ng/mL, and with both NE and cir-nDNA values higher than 21 and 9 ng/mL, respectively. Combining NE and cir-nDNA thresholds provides an AUC of 0.998 as compared to 0.953 and 0.940 in NE alone and cir-nDNA alone, respectively. While differences seem weak, they are of great importance when considering either a diagnostic kit or clinical routine test, as well as a screening test for inflammatory disease such as infectious, cancer or auto-immune diseases.

Data from the study described in example 17 on NS, S and Healthy individuals (HI) (Table 9) further illustrate the importance in combining NETs and cirDNA markers. For instance, when taking into consideration the analysis of NE, MPO and cir-nDNA and a threshold of 21, 21.5 and 9 ng/mL respectively:
the HI cohort shows 13, 13 and 13 false positives out of 119 subjects when independently taking NE, MPO and cir-nDNA values, respectively; whereas there are only 3 false positive when taking into consideration positivity when the three biomarkers are above the threshold meaning being all positive.
the NS cohort shows 2, 0 and 3 false positives out of 26 subjects when independently taking NE, MPO and cir-nDNA values, respectively; whereas there are 4 false negatives when taking into consideration positivity when the three biomarkers are above the threshold meaning being all positive.
the S cohort shows 2, 0 and 0 false positives out of 44 subjects when independently taking NE, MPO and cir-nDNA values, respectively; whereas there are only 2 false negatives when taking into consideration positivity when the three biomarkers are above the threshold meaning being all positive.

Note, when adding the MNR (threshold of 0.014) to the combination of the previous three biomarkers, the false positive rate reduced down to one false positive (vs three) out of 119 HI cohort, while false negative rate remains the same in NS (vs four) or in S (vs two) cohort. Consequently, combining NET and cirDNA markers using threshold or reference values critically improves test performance in diagnosing inflammatory diseases by greatly reducing the rate of false positives.

Determination of algorithms specifically designed for optimizing diagnosis based on this strategy is therefore a promising approach.

### REFERENCES

1. Thierry, A. & Roch, B. NETs By-products and Extracellular DNA May Play a Key Role in COVID-19 Pathogenesis: Incidence on Patient Monitoring and Therapy. https://www.preprints.org/manuscript/202004.0238/v1 (2020) doi:10.20944/preprints202004.0238.v1.
2. Barnes, B. J. et al. Targeting potential drivers of COVID-19: Neutrophil extracellular traps. J. Exp. Med. 217, e20200652 (2020).
3. Zuo, Y. et al. Neutrophil extracellular traps in COVID-19. JCI Insight (2020) doi:10.1172/jci.insight.138999.
4. Thierry, A. R. & Roch, B. SARS-CoV2 may evade innate immune response, causing uncontrolled neutrophil extracellular traps formation and multi-organ failure. Clin. Sci. 134, 1295-1300 (2020).
5. Thierry A R, Roch B. Neutrophil Extracellular Traps and By-Products Play a Key Role in COVID-19: Pathogenesis, Risk Factors, and Therapy. J Clin Med. 2020 Sep. 11;9(9):2942. doi: 10.3390/jcm9092942. PMID: 32933031; PMCID: PMC7565044
6. Thierry A R. Anti-protease Treatments Targeting Plasmin(ogen) and Neutrophil Elastase May Be Beneficial in Fighting COVID-19. Physiol Rev. 2020 Oct. 1;100(4):1597-1598. doi: 10.1152/physrev.00019.2020. PMID: 32639219; PMCID: PMC7365835.
7. Thierry A R. Host/genetic factors associated with COVID-19 call for precision medicine. Precision Clinical Medicine, Volume 3, Issue 3, September 2020, Pages 228-234, https://doi.org/10.1093/pcmedi/pbaa026
8. Zhang, B. et al. Clinical characteristics of 82 death cases with COVID-19. http://medrxiv.org/lookup/doi/10.1101/2020.02.26.20028191 (2020) doi:10.1101/2020.02.26.20028191.
9. Li, T., Lu, H. & Zhang, W. Clinical observation and management of COVID-19 patients. Emerg. Microbes Infect. 9, 687-690 (2020).
10. Brinkmann, V. Neutrophil Extracellular Traps in the Second Decade. J. Innate Immun. 10, 414-421 (2018).
11. Daniel, C. et al. Extracellular DNA traps in inflammation, injury and healing. Nat. Rev. Nephrol. 15, 559-575 (2019).
12. Euler, M. & Hoffmann, M. H. The double-edged role of neutrophil extracellular traps in inflammation. Biochem. Soc. Trans. BST20190629 (2019) doi:10.1042/BST20190629.
13. Schönrich, G. & Raftery, M. J. Neutrophil Extracellular Traps Go Viral. Front. Immunol. 7, (2016).
14. Costanza, M. et al. DNA threads released by activated CD4+ T lymphocytes provide autocrine costimulation. Proc. Natl. Acad. Sci. 116, 8985-8994 (2019).
15. Hirose, T. et al. Presence of Neutrophil Extracellular Traps and Citrullinated Histone H3 in the Bloodstream of Critically Ill Patients. PLoS ONE 9, e111755 (2014).
16. Sung, P.-S. & Hsieh, S.-L. CLEC2 and CLEC5A: Pathogenic Host Factors in Acute Viral Infections. Front. Immunol. 10, 2867 (2019).
17. Devaux, C. A., Rolain, J.-M., Colson, P. & Raoult, D. New insights on the antiviral effects of chloroquine against coronavirus: what to expect for COVID-19? Int. J. Antimicrob. Agents 105938 (2020) doi:10.1016/j.ijantimicag.2020.105938.
18. Wright, H. L., Cross, A. L., Edwards, S. W. & Moots, R. J. Effects of IL-6 and IL-6 blockade on neutrophil function in vitro and in vivo. Rheumatology 53, 1321-1331 (2014).
19. Thierry, A. R. Does the newly observed inflammatory syndrome in children demonstrate a link between uncontrolled neutrophil extracellular traps formation and COVID-19? Pediatr. Res. (2020) doi:10.1038/s41390-020-0996-1.
20. Grommes, J. & Soehnlein, O. Contribution of Neutrophils to Acute Lung Injury. Mol. Med. 17, 293-307 (2011).
21. Tan, K. S. et al. Respiratory Viral Infections in Exacerbation of Chronic Airway Inflammatory Diseases: Novel Mechanisms and Insights From the Upper Airway Epithelium. Front. Cell Dev. Biol. 8, 99 (2020).
22. Yoshida, Y. et al. Enhanced formation of neutrophil extracellular traps in Kawasaki disease. Pediatr. Res. (2020) doi:10.1038/s41390-019-0710-3.
23. Wichmann, D. et al. Autopsy Findings and Venous Thromboembolism in Patients With COVID-19: A Prospective Cohort Study. Ann. Intern. Med. M20-2003 (2020) doi:10.7326/M20-2003.
24. Ji, H.-L., Zhao, R., Matalon, S. & Matthay, M. A. Elevated Plasmin(ogen) as a Common Risk Factor for COVID-19 Susceptibility. Physiol. Rev. 100, 1065-1075 (2020).
25. Barbosa da Cruz, D. et al. DNA-bound elastase of neutrophil extracellular traps degrades plasminogen, reduces plasmin formation, and decreases fibrinolysis: proof of concept in septic shock plasma. FASEB J. 33, 14270-14280 (2019).
26. Meddeb, R., Pisareva, E. & Thierry, A. R. Guidelines for the Preanalytical Conditions for Analyzing Circulating Cell-Free DNA. Clin. Chem. 65, 623-633 (2019).
27. Thierry, A. R. et al. Clinical validation of the detection of KRAS and BRAF mutations from circulating tumor DNA. Nat. Med. 20, 430-435 (2014).
28. Meddeb, R. et al. Quantifying circulating cell-free DNA in humans. Sci. Rep. 9, 5220 (2019).
29. Templeton, A. J.; McNamara, M. G.; Šeruga, B.; Vera-Badillo, F. E.; Aneja, P.; Ocaña, A.; Leibowitz-Amit, R.; Sonpavde, G.; Knox, J. J.; Tran, B.; Tannock, I. F.; Amir, E. Prognostic Role of Neutrophil-to-Lymphocyte Ratio in Solid Tumors: A Systematic Review and Meta-Analysis. JNCI: Journal of the National Cancer Institute 2014, 106 (6). https://doi.org/10.1093/jnci/dju124.
30. Erpenbeck, L.; Schön, M. P. Neutrophil Extracellular Traps: Protagonists of Cancer Progression? Oncogene 2017, 36 (18), 2483-2490. https://doi.org/10.1038/onc.2016.406.
31. Munir, H.; Jones, J. O.; Janowitz, T.; Hoffmann, M.; Euler, M.; Martins, C. P.; Welsh, S. J.; Shields, J. D. Stromal-Driven and Amyloid β-Dependent Induction of Neutrophil Extracellular Traps Modulates Tumor Growth. Nat Commun 2021, 12 (1), 683.
32. Yang, L.; Liu, Q.; Zhang, X.; Liu, X.; Zhou, B.; Chen, J.; Huang, D.; Li, J.; Li, H.; Chen, F.; Liu, J.; Xing, Y.; Chen, X.; Su, S.; Song, E. DNA of Neutrophil Extracellular Traps Promotes Cancer Metastasis via CCDC25. Nature 2020, 583 (7814), 133-138. https://doi.org/10.1038/s41586-020-2394-6.
33. Nolan, E.; Malanchi, I. Neutrophil ‘Safety Net’ Causes Cancer Cells to Metastasize and Proliferate. Nature 2020, 583 (7814), 32-33. https://doi.org/10.1038/d41586-020-01672-3.
34. Wolach, O.; Sellar, R. S.; Martinod, K.; Cherpokova, D.; McConkey, M.; Chappell, R. J.; Silver, A. J.; Adams, D.; Castellano, C. A.; Schneider, R. K.; Padera, R. F.; DeAngelo, D. J.; Wadleigh, M.; Steensma, D. P.; Galinsky, I.; Stone, R. M.; Genovese, G.; McCarroll, S. A.; Iliadou, B.; Hultman, C.; Neuberg, D.; Mullally, A.; Wagner, D. D.; Ebert, B. L. Increased Neutrophil Extracellular Trap Formation Promotes Thrombosis in Myeloproliferative Neoplasms. Sci. Transl. Med. 2018, 10 (436), eaan8292. https://doi.org/10.1126/scitranslmed.aan8292.
35. Tambralli, A.; Gockman, K.; Knight, J. S. NETs in APS: Current Knowledge and Future Perspectives. Curr Rheumatol Rep 2020, 22 (10), 67. https://doi.org/10.1007/s11926-020-00936-1.
36. Abdel-Wahab, N.; Tayar, J. H.; Fa'ak, F.; Sharma, G.; Lopez-Olivo, M. A.; Yousif, A.; Shagroni, T.; Al-Hawamdeh, S.; Rojas-Hernandez, C. M.; Suarez-Almazor, M. E. Systematic Review of Observational Studies Reporting Antiphospholipid Antibodies in Patients with Solid Tumors. Blood Advances 2020, 4 (8), 1746-1755. https://doi.org/10.1182/bloodadvances.2020001557.
37. Sanchez, C.; Roch, B.; Mazard, T.; Blache, P.; Al Amir Dache, Z.; Pastor, B.; Pisareva, E.; Tanos, R.; Thierry, A. R. Circulating Nuclear DNA Structural Features, Origins, and Complete Size Profile Revealed by Fragmentomics. JCI Insight 2021. https://doi.org/10.1172/jci.insight.144561.
38. Tanos R, Tosato G, Otandault A, et al. Machine Learning-Assisted Evaluation of Circulating DNA Quantitative Analysis for Cancer Screening. Adv Sci. 2020;7(18):2000486. doi:10.1002/advs.202000486.
39. Medini H, Zirman A, Mishmar D. Immune system cells from COVID-19 patients display compromised mitochondrial-nuclear expression co-regulation and rewiring toward glycolysis. iScience. 2021;24(12):103471. doi:10.1016/j.isci.2021.103471.
40. Harzallah I, Debliquis A, Drénou B. Lupus anticoagulant is frequent in patients with Covid-19. J Thromb Hacmost. 2020;18(8):2064-2065. doi:10.1111/jth. 14867
41. Zuo Y, Estes S K, Ali R A, et al. Prothrombotic autoantibodies in serum from patients hospitalized with COVID-19. Sci Transl Med. 2020;12(570):eabd3876. doi:10.1126/scitranslmed.abd3876
42. Hollerbach A, Müller-Calleja N, Pedrosa D, et al. Pathogenic lipid-binding antiphospholipid antibodies are associated with severity of COVID-19. J Thromb Haemost. 2021;19(9):2335-2347. doi:10.1111/jth.15455.
43. Shi H, Zuo Y, Navaz S, et al. Endothelial Cell-Activating Antibodies in COVID-19. Infectious Diseases (except HIV/AIDS); 2021. doi:10.1101/2021.01.18.21250041.

44. Tambralli A. Gockman K. Knight J S. NETs in APS: Current Knowledge and Future Perspectives. Curr Rheumatol Rep. 2020;22(10):67. doi:10.1007/s11926-020-00936-1

1. A method for diagnosing a subject for an inflammatory disease, comprising:
i) determining in a sample obtained from the subject a level of at least one marker selected in the group consisting in NET protein markers, cir-nDNA, cir-mtDNA and/or a cir-DNA fragmentation index;
ii) comparing said level determined at step i) with a predetermined reference value;
iii) determining that the subject has an inflammatory disease when the level of the NET protein markers, cir-nDNA or cir-DNA fragmentation index determined at step i) is higher than the predetermined reference value or when the level of cir-mtDNA determined at step i) is lower than the predetermined reference value, and identifying the subject as being in need for treatment for the inflammatory disease; and
iv) determining that the subject does not have an inflammatory disease when the level of the NET protein markers, cir-nDNA or cir-nDNA fragmentation index determined at step i) is lower than the predetermined reference value or when the level of cir-mtDNA determined at step i) is higher than the predetermined reference value.

2. The method for diagnosing a subject for an inflammatory disease according to the claim 1 wherein the level of at least two markers selected in the group consisting in NET protein markers, cir-nDNA, cir-mtDNA and/or a cir-DNA fragmentation index are determined in step iii).

3. The method for diagnosing a subject for an inflammatory disease according to claim 1 wherein the NET protein markers are selected from the group consisting of NE (neutrophil elastase), MPO (myeloperoxidase), citrullinated histones, proteinase 3, cathepsin, lactoferrin, gelatinase, anti-phospholipid like anti-cardiolipin, and anti-phosphatidylserine.

4. A method for diagnosing a subject for an inflammatory disease, comprising: the steps of
i) determining in a sample obtained from the subject a level of long and short fragments of cir-nDNA,
ii) calculating a ratio of long over short fragments of cir-nDNA (DII),
iii) comparing said level determined at step i) with a predetermined reference value, and
iv) determining that the subject has an inflammatory disease when the calculated ratio determined at step ii) is higher than the predetermined reference value, and identifying the subject as being in need for treatment for the inflammatory disease, and
v) determining that the subject does not have an inflammatory disease when the calculated ratio determined at step i) is lower than the predetermined reference value.

5. The method for diagnosing a subject for an inflammatory disease according to the claim 4 wherein cir-nDNA fragments have a length between 67 bp and 305 bp.

6. The method for diagnosing a subject for an inflammatory disease according to the claim 5 wherein the cir-nDNA long fragments are higher than 200 pb or higher than 260 bp and the short fragments are lower than 150 pb or lower than 80 bp.

7. A method for diagnosing a subject for an inflammatory disease, comprising: the steps of
i) determining in a sample obtained from the subject a level of cir-mtDNA and cir-nDNA
ii) calculating a MNR ratio which is equal to the level of cir-mtDNA over the level of cir-nDNA,
iii) comparing said ratio determined at step ii) with a predetermined reference value,
iv) determining that the subject has an inflammatory disease when the calculated ratio determined at step ii) is lower than the predetermined reference value, and identifying the subject as being in need for treatment for the inflammatory disease, and
v) determining that the subject does not have an inflammatory disease when the calculated ratio determined at step ii) is higher than the predetermined reference value.

8. The method for diagnosing a subject for an inflammatory disease according to claim 1 wherein
the level of the elastase (NE), the myeloperoxidase (MPO) and Cir-nDNA are combined, or
the level of the elastase (NE), the myeloperoxidase (MPO) and Cir-mtDNA are combined, or
the level of the elastase (NE), the myeloperoxidase (MPO) and the MNR are combined, or
the level of the elastase (NE), the myeloperoxidase (MPO), Cir-nDNA and aCL are combined.

9. The method for diagnosing a subject for an inflammatory disease according to claim 1 wherein at least one of
the level of the elastase (NE) is higher than 2-fold as compared to reference value,
the level of the myeloperoxidase (MPO) is higher than 2-fold as compared to reference value,
the level of Cir-nDNA is higher than 2-fold as compared to reference value,
the level of Cir-mtDNA is lower than 2-fold as compared to reference value,
the level of the MNR is lower than 3-fold as compared to reference value, and
the level of aCL is higher by 1.5-fold, as compared to reference value.

10. The method for diagnosing a subject for an inflammatory disease according to claim 1 wherein the level of NE, MPO, cir-nDNA and MNR are combined.

11. The method for diagnosing a subject for an inflammatory disease according to claim 1 wherein threshold values for NE, MPO, cir-nDNA, cir-mtDNA, and MNR are respectively at least to 15 ng/ml, 20 ng/mL, 7 ng/ml, 0.1 ng/ml and 0.014, or are respectively at least 21 ng/ml, 21.5 ng/mL, 9 ng/ml, 0.06 ng/ml and 0.01.

12. A method for diagnosing a subject for an inflammatory disease comprising the steps of:
a. extracting the cir-DNA (cir-nDNA or cir-mtDNA) from a sample obtained from the subject;
b. determining the level of at least one single or double stranded DNA fragment having a length between 20 to 440 base pairs (bp);
c. comparing the level determined at step b) with a predetermined reference value; and
d. concluding that the subject suffers from an inflammatory disease when the level determined at step c) differ from the predetermined reference value.

13. A method for diagnosing a subject for an inflammatory disease comprises the steps of:
a. extracting a cir-DNA (cir-nDNA or cir-mtDNA) from a sample obtained from the subject;
b. determining a level of a first single or double stranded DNA fragment having a length between 20 to 440 bp;
c. determining a level of a second single or double stranded DNA fragment having a length between 20 to 440 bp;
d. calculating a ratio of the level determined at step b) to the level determined at step c) or alternatively a ratio of the level determined at step c) to the level determined at step b);
e. comparing the ratio determined at step d) with a predetermined corresponding reference value; and
f. concluding that the subject suffers from an inflammatory disease when the ratio determined at step d) differs from the predetermined corresponding reference value.

14. The method for diagnosing a subject for an inflammatory disease according to claim 13 further comprising that the length of the fragment is less than 90 bp, or is more than 167 bp, or is between 90 to 167 bp, or is between 142 to 152, or is between 167 to 220, or is between 220 to 440 bp.

15. The method for diagnosing a subject for an inflammatory disease according to claim 1 wherein the inflammatory disease is pathogen infection, an autoimmune disease or a cancer.

16. The method for diagnosing a subject for an inflammatory disease according to the claims 15 wherein the pathogen infection is the Covid-19.

17. The method for diagnosing a subject for an inflammatory disease according to the claims 15 wherein the cancer is a colorectal cancer or a metastatic colorectal cancer (mCRC).

18. The method for diagnosing a subject for an inflammatory disease according to the claims 15 wherein the autoimmune disease is a lupus.

19. A method for treating an inflammatory disease diagnosed by the method of claim 1 comprising administering to a subject in need thereof an anti-inflammatory disease treatment.

20. A kit for diagnosing an inflammatory disease, comprising means for determining a marker selected from the group consisting of NET protein markers, cir-nDNA or cir-mtDNA; and instructions for carrying out the method of claim 1.
